(19) United States
(12) Patent Application Publication
(10) Pub. No.: US 2025/0114412 A1
(43) Pub. Date: Apr. 10, 2025

(54) METHODS OF SYNBIOTIC TREATMENT TO IMPROVE HEALTH

(71) Applicant: SEED HEALTH INC., Venice, CA (US)

(72) Inventors: Ara KATZ, Venice, CA (US); Raja DHIR, Venice, CA (US); Braden TIERNEY, New York, NY (US)

(21) Appl. No.: 18/836,577

(22) PCT Filed: Feb. 7, 2022
(86) PCT No.: PCT/US22/15509

§ 371 Date: Aug. 7, 2024

(51) Int. Cl. A61K 35/745; A61K 31/702; A61K 31/733; A61K 35/747

(52) U.S. Cl. CPC A61K 35/745; A61K 31/702; A61K 31/733; A61K 35/747

(57) ABSTRACT

Synbiotic compositions including both a prebiotic component and a probiotic component are provided. The prebiotic component includes at least one compound that can be converted, by a microbial strain present in the healthy human gut microbiota, into a bioactive metabolite, and the probiotic component includes a rationally defined and assembled consortium of microbial strains.

### FIELD

The present disclosure is related generally to microbial biologics for pediatric use, and particularly to rationally defined microbial consortia that are effective to impart health across organ systems in human children by modulating the function of the native gut microbiota and host tissue.

### BACKGROUND

### Human Gut Microbiome

The human gut microbial community is a complex network of bacteria, archaea, viruses, fungi, and even protozoa that reside mainly in the colon of the host. This gut microbial community is comparable to the human body in the number of cells but contributes more than a 100-fold greater gene coding capacity.

Due to its important role in human health, the microbiome is sometimes considered an organ of the human body, and much like other organs, this microbial organ evolves temporally in terms of its microbial numbers and complexity. While microbial DNA has been detected at the fetal stage, the gut may be considered sterile or at best sparsely populated at birth. Further colonization of the gut depends on the type of birth, the environment, and the first food, i.e., breast milk or formula. During the first few days of life, the gut microbiome is dominated by aerobes and facultative anaerobes such as enterobacteria, enterococci, staphylococci, and streptococci. These microbes proliferate in the gut, consequently lowering the intestinal oxygen concentrations sufficiently to enable the proliferation of obligate anaerobes such as Bifidobacterium spp., which then become the predominant colonizer in the first few weeks. In some infants, another type of facultative anaerobe, Bacteroides spp., may also predominate. As more foods are introduced to the infant's diet around six months of age, the infant acquires diverse bacteria, resulting in a more complex microbiome by twelve months of age. During this period there is an increase in numbers of clostridia, commensurate with an increased need to digest a greater variety of microbiota-accessible carbohydrates that reach the colon, resulting in a greater diversity and maturation of the infant gut microbiome. By about three years of age, a child's gut microbiome resembles that of an adult. The members of the mature gut microbial community are mainly from five major phyla—Firmicutes, Bacteroidetes, Actinobacteria, Proteobacteria, and Verrucomicrobia—with the former two comprising about 90% of the population.

Gut bacteria have co-evolved with their human host, for the purpose of extracting energy for their own existence, while also nurturing the host. Gut microbiota play key roles in digestion of nutrients, maturation of the gastrointestinal tract, immune activation, protection against pathogens, generation of vitamins that are otherwise not produced by the host, and even brain health. A dysbiosis in gut microbiota is associated with gut-related diseases, such as inflammatory bowel disease (IBD) and irritable bowel syndrome (IBS); immune diseases, such as asthma, atopic dermatitis, and rheumatoid arthritis; and lifestyle-associated metabolic diseases, such as obesity and diabetes. Gut bacteria are also increasingly associated with brain-related issues, such as mood, anxiety, and neurodegenerative disorders.

The gut commensals are adept at cooperatively extracting energy from the organic material that escapes metabolism by the host and reaches the colon. Thus, undigested food remnants—mainly plant food fiber and polyphenols, as well as glycoproteins sloughed off from intestinal mucosa—are metabolized by gut bacteria to generate acid metabolites. Chief among the acid metabolites are lactate, an organic acid, and short-chain fatty acids (SCFAs) such as acetate, propionate, and butyrate.

Acetate and lactate are generated in the earlier steps of carbohydrate degradation by bacteria. Then, by a process of mutualistic resource sharing, these metabolites serve as carbon sources for propionate and butyrate producers, which can thus be called “secondary” feeders. While acetate is produced by most gut bacteria, lactate is produced by a smaller set of bacteria, particularly Bifidobacterium spp. and lactic acid bacteria (order Lactobacillus).

SCFAs play major roles in regulating host metabolism, immune defense, and even brain health. The three-carbon molecule acetate beneficially affects host energy and metabolism via appetite modulation and fat oxidation. Propionate is known to increase plasma glucagon, fatty acid binding protein 4 (FABP4), and norepinephrine levels, leading to insulin resistance and compensatory hyperinsulinemia. Butyrate provides the majority of fuel required for the colonic cells and plays a key role in maintaining the gut barrier by modulating the tight junction proteins. Butyrate also acts as a histone deacetylase inhibitor and affects signaling through several G protein-coupled receptors that mediate anti-inflammatory activity. Maintenance of the barrier is energy-intensive and the microbially-generated butyrate is a chief source of this energy. Collectively, butyrate has beneficial effects on intestinal homeostasis and energy metabolism, and its anti-inflammatory action enhances intestinal barrier function and mucosal immunity.

The balanced presence of SCFAs exerts an essential role in preventing chronic inflammatory states, inhibiting the production of pro-inflammatory cytokines, regulating T cells, and protecting colonic epithelial cells. The influence of SCFAs extends beyond the gastrointestinal tract; these metabolites link to the respiratory system by inhibiting pulmonary innate lymphoid cell (ILC2) functions that stimulate airway hyperreactivity, inhibit skin pathogens through the gut-skin axis, and are regulators of bone mass and osteoclast metabolism. Furthermore, SCFAs also act via the gut-brain axis to regulate mood, sleep, and neuronal health.

### Gut Microbiome and Health in Childhood

The connection between the gut microbiome and health is becoming increasingly evident. One of the earliest associations was well-described by the “hygiene hypothesis,” which suggested that infections early in life by “unhygienic contact” (e.g., due to living on a farm, having pets, etc.) prevented allergies. A definite association has been made between gut microbiota and development of disease in children.

There are many factors that affect gut microbial development and maturation in children. These factors include birth mode, first food (i.e., breast milk vs. formula), household size, and environment (location, urban vs. rural, presence of pets, etc.). Non-limiting examples of associations between gut microbiome status and disease in children include an association between allergy and decreased species diversity; associations between autism spectrum disorder and increased clostridial species and/or Sutterella and Desulfovibrio species; associations between colic and decreased microbial diversity and/or increased anaerobic bacterial populations; an association between the initiation of eczema and early colonization with opportunistic species; associations between malnutrition and anaerobic depletion, early dysbiosis, and/or intestinal pathogenic overabundance with decreased bacterial diversity; association between prematurity and increased proteobacteria and/or decreased microbial diversity; an association between sepsis and altered microbiota structure and composition prior to disease onset; associations between type I diabetes and increased Bacteriodetes:Firmicutes ratio, increased Clostridia species, decreased butyrate-producing bacteria, decreased bacterial diversity, and/or decreased microbial community stability; and associations between type II diabetes and increased Firmicutes: Bacteroidetes ratio and/or increased SCFAs.

### Probiotics

It has been over a century since the correlation between enhanced longevity and the consumption of yogurt containing lactic acid-producing bacteria was first observed, and since Bifidobacterium spp. were first detected in the feces of breastfed infants but not of those who were formula-fed and suffering from diarrhea. These early “bacteria for health” concepts have culminated in the widely adopted use of the term “probiotics” to mean “live microorganisms that, when administered in adequate amounts, confer a health benefit on the host”.

Probiotics historically encompassed the genera Bifidobacterium and Lactobacillus. Over 250 Lactobacillus species were identified, which appeared to be highly genetically distinct from one another but also metabolically, ecologically, and functionally diverse. These observations led to a recent reclassification of the genus Lactobacillus into 25 genera, including the emended genus Lactobacillus (which encompasses host-adapted organisms that are now referred to as the Lactobacillus delbrueckii group), Paralactobacillus, and 23 novel genera.

The National Institutes of Health Human Microbiome Project showed great variation in the diversity and abundance of microbes among healthy individuals, indicating that there is no universally healthy microbiome. Despite this variety of healthy states, important correlations between health and disease status and the presence or abundance of specific microbial species have been established, raising the possibility that manipulation of these communities can prevent and treat illnesses. This concept fostered the isolation of specific microbiota members from healthy individuals for the development of so-called live biotherapeutic products (LBPs). These added to the classic lactobacilli- and Bifidobacterium-based probiotic products, aiming to beneficially impact disease status. Notably, gut microbiota interventions not only bring health benefits to the local environment but also impact distantly, through for instance the gut-skin, gut-heart, and gut-brain axes.

### Prebiotics

Prebiotics have been defined by the International Scientific Association for Probiotics and Prebiotics as “substrates that are selectively utilized by host microorganisms conferring a health benefit.” For a substrate to be considered prebiotic under this definition, it must meet three criteria: (1) it is not digested or utilized by systems of the host; (2) it is selectively utilized by host microbes, resulting in a favorable change in the microbial balance or in the formation of metabolites that benefit the host; and (3) the benefits to the host have been demonstrated by clinical studies and are a result of microbial changes in the host gut.

The primary types of dietary prebiotics are certain microbiota-fermentable dietary fibers. Some plant-derived polyphenols can also demonstrate prebiotic effects due to their ability to selectively modulate the growth of bacteria and undergo microbial biotransformation into a range of metabolites with beneficial effects on the host.

Dietary fiber prebiotics are polymeric and oligomeric carbohydrates that are utilized by gut microbes, resulting in the production of beneficial metabolites through a multistep fermentation process. Most commercial prebiotics today fall into this category; common examples include fructo-oligosaccharides (FOSs), galacto-oligosaccharides (GOSs), and inulin. The number of fructose molecules linked to each other (i.e., the degree of polymerization, or DP) and the branching of these molecules play an important role in the fermentability of these prebiotics. In general, the low-DP (DP of 1 or 2) fructans are broken down more readily and more rapidly by gut bacteria in laboratory models of the gut, while large-DP fructans (DP of 3 to 60) exhibit a slower, steadier rate of fermentation by fecal bacteria. Thus, a higher-DP inulin may remain intact long enough to be subjected to intestinal peristaltic forces and pushed more distally into the colon, enabling an extension of the duration of microbial activity.

### Synbiotics

When both probiotics and prebiotics are combined in a product, the product may be referred to as a “synbiotic.” A synbiotic may therefore be defined as “a mixture containing live organisms and substrate(s) selectively utilized by host microorganisms that confers a health benefit on the host.” Such a formulation may be complementary in its mechanism of action, i.e., the prebiotic targets the autochthonous commensals, or synergistic, in that the prebiotic substrate is selectively utilized by the co-administered probiotics. Delivery of synbiotics to targeted niches in the human gastrointestinal tract is challenging due to the harsh and changing conditions of the gut, issues related to maintenance of activity and efficacy of the probiotic, and masking unpleasant flavors of the prebiotic. Synbiotic delivery technologies have been, and continue to be, extensively researched by the functional food and supplement industry.

### SUMMARY

In an aspect of the present disclosure, a method for treating a disease or achieving a physiological objective in a human subject comprises administering to the subject a therapeutically effective amount of a synbiotic composition, the synbiotic composition comprising a prebiotic component, comprising at least one compound that can be converted, by a microbial strain present in the healthy human gut microbiota, into a bioactive metabolite; and a probiotic component, comprising a consortium of microbial strains, the consortium comprising at least two microbial strains selected from the group consisting of (i) Lacticaseibacillus rhamnosus SD-GG-BE; (ii) Ligilactobacillus salivarius SD-LS1-IT; (iii) Bifidobacterium breve SD-BR632-IT; (iv) Bifidobacterium breve SD-BR03-IT; (v) Bifidobacterium longum SD-CECT7347-SP; (vi) Lacticaseibacillus casei SD-CECT9104-SP; and (vii) Bifidobacterium lactis SD-CECT8145-SP.

In embodiments, the disease may be selected from the group consisting of adrenal leukodystrophy, AGE-induced genome damage, Alexander Disease, alopecia areata, Alper's Disease, Alzheimer's disease, amyotrophic lateral sclerosis, angina pectoris, arthritis, asthma, Balo's concentric sclerosis, Behcet's disease, bullous pemphigoid, Canavan disease, cardiac insufficiency including left ventricular insufficiency, central nervous system vasculitis, Charcot-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, herpes zoster viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, paraneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinitis pigmentosa, sarcoidosis, Schilder's Disease, subacute necrotizing myelopathy, susac syndrome, transplantation rejection, transverse myelitis, a tumor, ulcerative colitis, and Zellweger's syndrome.

In embodiments, the disease may be a gastroenterological or infectious disease. The disease may, but need not, be selected from the group consisting of irritable bowel syndrome, COVID-19, and constipation. The disease may, but need not, be antibiotic-induced dysbiosis of the subject's gut microbiota.

In embodiments, the disease may be selected from the group consisting of metabolic syndrome, type 2 diabetes, and pre-diabetes.

In embodiments, the physiological objective may be selected from the group consisting of improving cardiovascular health, maintaining or reducing body weight, reducing glycated hemoglobin levels, normalizing glucose or insulin response, treating acne or otherwise improving skin health, and improving cognitive function.

In embodiments, the method may further comprise co-administering, or administering in combination with the synbiotic composition, one or more other pharmaceutical formulations to a human subject, and the co-administration or administration in combination may improve a therapeutic effect of the one or more other pharmaceutical formulations relative to independent administration of the synbiotic composition and the one or more other pharmaceutical formulations.

In embodiments, the physiological objective may be selected from the group consisting of increasing Bristol Stool Form Scale (BSFS), decreasing duration of a bowel movement, relieving abdominal pain, relieving bloating, relieving heartburn, relieving acid reflux, relieving indigestion, maintaining or increasing diversity of the gastrointestinal microbiota, and improving health-related quality of life as measured by KINDL questionnaire score.

In embodiments, the synbiotic composition may be administered as an ingestible formulation. The ingestible formulation may, but need not, be in the form of a free-flowing powder provided in a single-serving sachet. The sachet may, but need not, comprise the prebiotic component in an amount of from about 1 mg to about 12 g, or from about 250 mg to about 11.75 g, or from about 500 mg to about 11.5 g, or from about 750 mg to about 11.25 g, or from about 1 g to about 11 g, or from about 1.25 g to about 10.75 g, or from about 1.5 g to about 10.5 g, or from about 1.75 g to about 10.25 g, or from about 2 g to about 10 g, or from about 2.25 g to about 9.75 g, or from about 2.5 g to about 9.5 g, or from about 2.75 g to about 9.25 g, or from about 3 g to about 9 g, or from about 3.25 g to about 8.75 g, or from about 3.5 g to about 8.5 g, or from about 3.75 g to about 8.25 g, or from about 4 g to about 8 g, or from about 4.25 g to about 7.75 g, or from about 4.5 g to about 7.5 g, or from about 4.75 g to about 7.25 g, or from about 5 g to about 7 g, or from about 5.25 g to about 6.75 g, or from about 5.5 g to about 6.5 g, or from about 5.75 g to about 6.25 g, or about 6 g. The sachet may, but need not, comprise the consortium of microbial strains in an amount of from about 62.5 million AFU to about 312.5 billion AFU, from about 625 million AFU to about 250 billion AFU, from about 1.25 billion AFU to about 125 billion AFU, from about 6.25 billion AFU to about 62.5 billion AFU, from about 12.5 billion AFU to about 60 billion AFU, from about 25 billion AFU to about 55 billion AFU, from about 40 billion AFU to about 50 billion AFU, or about 45 billion AFU. A material of the sachet may, but need not, have a moisture vapor transmission rate, at 23° C. and 50% relative humidity, of less than about 0.01 grams per square meter per day. A dose of the synbiotic composition may, but need not, be administered at least once per day, and a dose may, but need not, comprise the contents of one sachet. The sachet may, but need not, further comprise at least one pharmaceutically acceptable vehicle.

In embodiments, the synbiotic composition may be administered at least once per day for at least about 7 days.

In embodiments, the at least one compound that can be converted, by a microbial strain present in the healthy human gut microbiota, into a bioactive metabolite may comprise at least one fructan. The at least one fructan may, but need not, comprise at least one polysaccharide fructan. The at least one polysaccharide fructan may, but need not, comprise an inulin. The inulin may, but need not, be derived or extracted from at least one plant selected from the group consisting of agave, asparagus, banana, barley, burdock, camas, chicory, coneflower, costus, dandelion, elecampane, garlic, globe artichoke, Jerusalem artichoke, jicama, leek, leopard's bane, mugwort, onion, plantain, wheat, yacón, and yam. The at least one fructan may, but need not, comprise a fructooligosaccharide. The fructooligosaccharide may, but need not, be derived or extracted from at least one plant selected from the group consisting of agave, asparagus, banana, barley, burdock, camas, chicory, coneflower, costus, dandelion, elecampane, garlic, globe artichoke, Jerusalem artichoke, jicama, leek, leopard's bane, mugwort, onion, plantain, wheat, yacón, and yam.

In embodiments, the prebiotic component may consist essentially of about 50 wt % inulin and about 50 wt % fructooligosaccharides.

In embodiments, the consortium may comprise at least three, at least four, at least five, at least six, or all of (i) through (vii).

In embodiments, the consortium may further comprise at least one microbial strain selected from the group consisting of (viii) Lactobacillus acidophilus SD-NCFM-US; and (ix) Bifidobacterium animalis subsp. lactis SD-BI07-US. The consortium may, but need not, comprise both of (viii) and (ix). The consortium may, but need not, comprise all of (i) through (vii) and both of (viii) and (ix). The consortium may, but need not, consist essentially of (i) through (vii), (viii), and (ix).

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to about 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIGS. 1A, 1B, 1C, 1D, 1E, IF, 1G, 1H, and 1I are graphs of change in OD₆₀₀ of probiotic strains according to the present disclosure with various prebiotic substrates.

FIG. 2 is a plot of a fluorescence-activated cell sorting (FACS)-based determination of viability of cells of probiotic strains according to the present disclosure.

FIG. 3 is a schematic of a standard setup of a simulator of the human intestinal microbial ecosystem (SHIME).

FIG. 4 is a general schematic of an adapted SHIME system used to study survival of probiotic strains according to the present disclosure in the gastrointestinal tract (GIT).

FIGS. 5A and 5B are graphs of pH profile during survival experiments under fasted and fed conditions, respectively.

FIGS. 6A and 6B are graphs of average viable bacterial population during passage through the upper GIT under fasted and fed conditions, respectively, of naked probiotic strains according to the present disclosure.

FIGS. 7A and 7B are graphs of average viable bacterial population during passage through the upper GIT under fasted and fed conditions, respectively, of microencapsulated probiotic strains according to the present disclosure.

FIGS. 8A and 8B are graphs of average pH change during short-term colonic incubation of a predigested blank control or a microencapsulated probiotic composition according to the present disclosure when supplemented with non-sterile (FIG. 8A) or sterile (FIG. 8B) inoculum of a healthy child of 6-10 years of age.

FIGS. 9A and 9B are graphs of average lactate concentration obtained during short-term colonic incubation of a predigested blank control or a microencapsulated probiotic composition according to the present disclosure when supplemented with non-sterile (FIG. 9A) or sterile (FIG. 9B) inoculum of a healthy child of 6-10 years of age.

FIGS. 10A and 10B are graphs of average short-chain fatty acid (SCFA) production during short-term colonic incubation of a predigested blank control or a microencapsulated probiotic composition according to the present disclosure when supplemented with non-sterile (FIG. 10A) or sterile (FIG. 10B) inoculum of a healthy child of 6-10 years of age.

FIGS. 11A and 11B are graphs of average acetate production during short-term colonic incubation of a predigested blank control or a microencapsulated probiotic composition according to the present disclosure when supplemented with non-sterile (FIG. 11A) or sterile (FIG. 11B) inoculum of a healthy child of 6-10 years of age.

FIGS. 12A and 12B are graphs of average propionate production during short-term colonic incubation of a predigested blank control or a microencapsulated probiotic composition according to the present disclosure when supplemented with non-sterile (FIG. 12A) or sterile (FIG. 12B) inoculum of a healthy child of 6-10 years of age.

FIGS. 13A and 13B are graphs of average butyrate production during short-term colonic incubation of a predigested blank control or a microencapsulated probiotic composition according to the present disclosure when supplemented with non-sterile (FIG. 13A) or sterile (FIG. 13B) inoculum of a healthy child of 6-10 years of age.

FIGS. 14A and 14B are graphs of average branched short-chain fatty acid (bSCFA) production during short-term colonic incubation of a predigested blank control or a microencapsulated probiotic composition according to the present disclosure when supplemented with non-sterile (FIG. 14A) or sterile (FIG. 14B) inoculum of a healthy child of 6-10 years of age.

FIGS. 15A, 15B, 15C, and 15D are graphs of average viable bacterial population during passage through the upper GIT of a Culturelle® Kids Probiotic composition, a Garden of Life® Raw Probiotics Kids composition, an Optibac Probiotics® Babies & Children composition, and a Life-Space® Probiotic Powder for Children composition, respectively.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

“CRISPR” (Clustered Regularly Interspaced Short Palindromic Repeats) loci refers to certain genetic loci encoding components of DNA cleavage systems, for example, used by bacterial and archaeal cells to destroy foreign DNA. A CRISPR locus can consist of a CRISPR array, comprising short direct repeats (CRISPR repeats) separated by short variable DNA sequences (called spacers), which can be flanked by diverse Cas (CRISPR-associated) genes. The CRISPR-Cas system, an example of a pathway that was unknown to science prior to the DNA sequencing era, is now understood to confer bacteria and archaea with acquired immunity against phage and viruses. Intensive research over the past decade has uncovered the biochemistry of this system. CRISPR-Cas systems consist of Cas proteins, which are involved in acquisition, targeting and cleavage of foreign DNA or RNA, and a CRISPR array, which includes direct repeats flanking short spacer sequences that guide Cas proteins to their targets. Class 2 CRISPR-Cas are streamlined versions in which a single Cas protein bound to RNA is responsible for binding to and cleavage of a targeted sequence. The programmable nature of these minimal systems has facilitated their use as a versatile technology that is revolutionizing the field of genome manipulation.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus, and include adaptation molecules as well as interference molecules. An interference molecule of a bacterial adaptive immunity complex includes endonucleases. A Cas endonuclease described herein comprises one or more nuclease domains. A Cas endonuclease includes but is not limited to: the novel Cas-alpha protein disclosed herein, a Cas9 protein, a Cpf1 (Cas12) protein, a C2c1 protein, a C2c2 protein, a C2c3 protein, Cas3, Cas3-HD, Cas 5, Cas7, Cas8, Cas10, or combinations or complexes of these. A Cas protein may be a “Cas endonuclease” or “Cas effector protein”, that when in complex with a suitable polynucleotide component, is capable of recognizing, binding to, and optionally nicking or cleaving all or part of a specific polynucleotide target sequence.

CRISPR-Cas systems have been classified according to sequence and structural analysis of components. Multiple CRISPR/Cas systems have been described including Class 1 systems, with multisubunit effector complexes (comprising type I, type III, and type IV), and Class 2 systems, with single protein effectors (comprising type II, type V, and type VI). A CRISPR-Cas system comprises, at a minimum, a CRISPR RNA (crRNA) molecule and at least one CRISPR-associated (Cas) protein to form crRNA ribonucleoprotein (crRNP) effector complexes. CRISPR-Cas loci comprise an array of identical repeats interspersed with DNA-targeting spacers that encode the crRNA components and an operon-like unit of cas genes encoding the Cas protein components. The resulting ribonucleoprotein complex recognizes a polynucleotide in a sequence-specific manner. The crRNA serves as a guide RNA for sequence specific binding of the effector (protein or complex) to double strand DNA sequences, by forming base pairs with the complementary DNA strand while displacing the noncomplementary strand to form a so-called R-loop. RNA transcripts of CRISPR loci (pre-crRNA) are cleaved specifically in the repeat sequences by CRISPR associated (Cas) endoribonucleases in type I and type III systems or by RNase III in type II systems. The number of CRISPR-associated genes at a given CRISPR locus can vary between species.

Different cas genes that encode proteins with different domains are present in different CRISPR systems. The cas operon comprises genes that encode for one or more effector endonucleases, as well as other Cas proteins. Some domains may serve more than one purpose, for example Cas9 comprises domains for endonuclease functionality as well as for target cleavage, among others. The Cas endonuclease is guided by a single CRISPR RNA (crRNA) through direct RNA-DNA base-pairing to recognize a DNA target site that is in close vicinity to a protospacer adjacent motif (PAM). Class I CRISPR-Cas systems comprise Types I, III, and IV. A characteristic feature of Class I systems is the presence of an effector endonuclease complex instead of a single protein. A Cascade complex comprises a RNA recognition motif (RRM) and a nucleic acid-binding domain that is the core fold of the diverse RAMP (Repeat-Associated Mysterious Proteins) protein superfamily.

Type I CRISPR-Cas systems comprise a complex of effector proteins, termed Cascade (CRISPR-associated complex for antiviral defense) comprising at a minimum Cas5 and Cas7. The effector complex functions together with a single CRISPR RNA (crRNA) and Cas3 to defend against invading viral DNA. Type I systems are divided into seven subtypes.

Type III CRISPR-Cas systems, comprising a plurality of cas7 genes, target either ssRNA or ssDNA, and function as either an RNase as well as a target RNA-activated DNA nuclease. Type IV systems, although comprising typical type I cas5 and cas7 domains in addition to a cas8-like domain, may lack the CRISPR array that is characteristic of most other CRISPR-Cas systems.

Class II CRISPR-Cas systems comprise Types II, V, and VI. A characteristic feature of Class II systems is the presence of a single Cas effector protein instead of an effector complex. Types II and V Cas proteins comprise an RuvC endonuclease domain that adopts the RNase H fold. Type II CRISPR/Cas systems employ a crRNA and tracrRNA (trans-activating CRISPR RNA) to guide the Cas endonuclease to its DNA target. The crRNA comprises a spacer region complementary to one strand of the double strand DNA target and a region that base pairs with the tracrRNA (trans-activating CRISPR RNA) forming a RNA duplex that directs the Cas endonuclease to cleave the DNA target, leaving a blunt end. Spacers are acquired through a not fully understood process involving Cas1 and Cas2 proteins. Type II CRISPR/Cas loci typically comprise cas1 and cas2 genes in addition to the cas9 gene. Type II CRISR-Cas loci can encode a tracrRNA, which is partially complementary to the repeats within the respective CRISPR array, and can comprise other proteins such as Csn1 and Csn2. The presence of cas9 in the vicinity of cas1 and cas2 genes is the hallmark of type II loci. Type V CRISPR/Cas systems comprise a single Cas endonuclease, including Cpf1 (Cas12) that is an active RNA-guided endonuclease that does not necessarily require the additional trans-activating CRISPR (tracr) RNA for target cleavage, unlike Cas9. Type VI CRISPR-Cas systems comprise a cas13 gene that encodes a nuclease with two HEPN (Higher Eukaryotes and Prokaryotes Nucleotide-binding) domains but no HNH or RuvC domains, and are not dependent upon tracrRNA activity. The majority of HEPN domains comprise conserved motifs that constitute a metal-independent endoRNase active site. Because of this feature, it is thought that type VI systems act on RNA targets instead of the DNA targets that are common to other CRISPR-Cas systems.

To comply with written description and enablement requirements, incorporated herein by the following references are the following patent publications: 2014/0349405 to Sontheimer; 2014/0377278 to Elinav; 2014/0068797 to Doudna; 20200190494 to Hou, et. al.; and 2020/0199555 to Zhang.

To further comply with applicable written description and enablement requirements, incorporated herein by reference is U.S. Patent Application Publication 2020/0138722, entitled “Targeted gastrointestinal tract delivery of probiotic organisms and/or therapeutic agents,” published 7 May 2020 to Kabadi et al.

To further comply with applicable written description and enablement requirements, the following references are incorporated herein by reference:
PCT Application PCT/US2021/015103, entitled “Probiotic compositions and microbial consortia to improve human organ system health,” filed 26 Jan. 2021.
PCT Application PCT/US2021/015107, entitled “Methods and compositions for precision release of probiotics to improve human health,” filed 26 Jan. 2021.
U.S. Provisional Patent Application 63/141,874, entitled “Methods of probiotic treatment to improve human health,” filed 26 Jan. 2021.

As used herein, unless otherwise specified, the term “animal” refers to any organism of the kingdom Animalia, including but not limited to a human.

As used herein, unless otherwise specified, the term “disease” refers to a disease, disorder, or condition or a symptom thereof.

As used herein, unless otherwise specified, the term “ingestible formulation” refers to a composition of matter that is adapted or configured to be consumed by an animal by taking the composition in through the mouth into the gastrointestinal tract, for example by eating or drinking. An “ingestible composition” as that term is used herein may be provided in a form selected from the group consisting of ampules, aqueous solutions, aqueous suspensions, capsules, drops, granules, liquids, mists, powders, sachets, syrups, tablets, functionalized foods, beverages, toothpastes, sublingual articles, and the like.

As used herein, unless otherwise specified, the term “lactobacilli,” when used without further elaboration, refer to any organism that was, prior to the 2020 reclassification of the genus Lactobacillus into 25 distinct genera, classified as Lactobacillaceae.

As used herein, unless otherwise specified, the term “patient” refers to a mammal, including, by way of non-limiting example, a human.

As used herein, unless otherwise specified, the term “pharmaceutically acceptable” means approved or approvable by a regulatory agency of the Federal or a state government or listed in the U.S. Pharmacopoeia or other generally recognized pharmacopoeia for use in animals, and more particularly in humans.

As used herein, unless otherwise specified, the term “pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a substance provided by the present disclosure may be administered to a patient, which does not destroy the pharmacological activity thereof, and which is non-toxic when administered in doses sufficient to provide a therapeutically effective amount of the substance.

As used herein, unless otherwise specified, the term “pharmaceutical formulation” refers to a therapeutically active substance and at least one pharmaceutically acceptable vehicle, with which the substance is administered to a patient.

As used herein, unless otherwise specified, the term “prebiotic” refers to a substrate that is selectively utilized by a microorganism that confers a health benefit upon the microorganism's host.

As used herein, unless otherwise specified, the term “probiotic” refers to live microorganisms that, when administered in adequate amounts, confer a health benefit on the host.

As used herein, unless otherwise specified, the term “synbiotic” refers to a combination or mixture of probiotics and prebiotics that beneficially affects a host by improving the survival and implantation of live microbial dietary supplements in the gastrointestinal tract via selective stimulation of the growth, and/or activation of the metabolism, of one or more health-promoting microbes.

As used herein, the terms “treating” and “treatment” refer to reversing, alleviating, arresting, ameliorating, or preventing a disease or at least one of the clinical symptoms of a disease, reducing the risk of acquiring a disease or at least one of the clinical symptoms of a disease, inhibiting the progress of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease even though that patient does not yet experience or display symptoms of the disease.

As used herein, unless otherwise specified, the term “therapeutically effective amount” refers to the amount of a substance that, when administered to a subject for treating a disease, or at least one of the clinical symptoms of a disease, is sufficient to affect such treatment of the disease or symptom thereof. The “therapeutically effective amount” may vary depending, for example, on the substance, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

As used herein, unless otherwise specified, the term “therapeutically effective dose” refers to a dose that provides effective treatment of a disease or disorder in a patient. A therapeutically effective dose may vary from substance to substance, and from patient to patient, and may depend upon factors such as the condition of the patient and the route of delivery. A therapeutically effective dose may be determined in accordance with routine pharmacological procedures known to those skilled in the art.

The present disclosure provides rationally defined and assembled microbial consortia to impart health across organ systems by first modulating the function of the native gut microbiota and host tissue. More specifically, the disclosure provides designed oral prophylactic probiotic strain mixtures to maintain and improve host health status in local and distant body sites.

In embodiments, the present disclosure provides one or more consortia of live microorganisms with a range of benefits verified in humans. Viability of the synbiotic compositions of the present disclosure throughout production and administration has been validated by comprehensive evaluation of Active Fluorescent Units (AFUs) in a simulated human intestinal environment, as described in further detail in the Examples provided below. In addition to extensive mechanistic and genetic characterization, the consortia include strains that have been clinically evaluated in strain-specific, double-blind, placebo-controlled human studies for clinical outcomes including improvement in digestion, modulation of the gut-skin axis, low-density lipoprotein modulation, gut immunological response, epithelial barrier regulation, and micronutrient synthesis.

### Microbial Strains

The development of next-generation sequencing (NGS) technologies has introduced new and much cheaper methods for prokaryotic whole genome sequencing. In combination with the decreasing price for computational resources needed for bioinformatic analyses, this has resulted in the publication of a large number of new bacterial genomes. More and more “non-bioinformatic” labs are now sequencing their own prokaryotic genomes and facing questions such as which sequencing platform to choose, how many libraries should be generated, which assembly method should be used for the libraries, etc.

Embodiments of the present disclosure include synbiotic compositions comprising Lacticaseibacillus rhamnosus SD-GG-BE. This bacterial strain may have any one or more of several beneficial effects on the health of the host. By way of first non-limiting example, Lacticaseibacillus rhamnosus SD-GG-BE may reduce toll-like receptor mRNA levels of antigen-presenting cells (APCs), reduce CD16 expression in macrophages and CD11 expression in monocytes, and/or induce type-1 immune response polarization as observed from elevated production of IL-12 and TNF-α. By way of second non-limiting example, Lacticaseibacillus rhamnosus SD-GG-BE may reduce abdominal pain in children with functional dyspepsia, irritable bowel syndrome, and/or functional abdominal pain. By way of third non-limiting example, Lacticaseibacillus rhamnosus SD-GG-BE may improve bloody stool, diarrhea, restiveness, abdominal distention, mucus in stool, and/or vomiting in children with a cow milk protein allergy. By way of fourth non-limiting example, Lacticaseibacillus rhamnosus SD-GG-BE may shorten the duration of diarrhea and/or reduce the daily number of stools in children with diarrhea. By way of fifth non-limiting example, Lacticaseibacillus rhamnosus SD-GG-BE may reduce the effects of antibiotic treatment on the gut microbiota of children. By way of sixth non-limiting example, Lacticaseibacillus rhamnosus SD-GG-BE may play a role in preventing or treating infant colic.

Embodiments of the present disclosure include synbiotic compositions comprising Ligilactobacillus salivarius SD-LS1-IT. This bacterial strain may have any one or more of several beneficial effects on the health of the host. By way of first non-limiting example, Ligilactobacillus salivarius SD-LS1-IT may improve clinical parameters of atopic dermatitis as measured by SCORAD and Dermatology Life Quality (DLQ) index (particularly in children), reduce microbial translocation and/or immune activation, and/or improve T-helper cell (Th) 17/regulatory T cell (T_{reg}) and Th1/Th2 ratios. By way of second non-limiting example, Ligilactobacillus salivarius SD-LS1-IT may decrease staphylococci count in the host's feces. By way of third non-limiting example, Ligilactobacillus salivarius SD-LS1-IT may improve clinical parameters of atopic dermatitis as measured by itch index, which may persist after cessation of synbiotic administration. By way of fourth non-limiting example, Ligilactobacillus salivarius SD-LS1-IT may decrease staphylococcal load and/or reduce production of Th2 cytokines and maintain stable production of Th1 cytokines. By way of fifth non-limiting example, Ligilactobacillus salivarius SD-LS1-IT may reduce pro-inflammatory cytokines, increase anti-inflammatory cytokines, inhibit reactive oxygen species production, restore cellular membrane integrity, and/or inhibit pathogenic E. coli and Klebsiella pneumoniae.

Embodiments of the present disclosure include synbiotic compositions comprising Bifidobacterium breve SD-B632-IT. This bacterial strain may have any one or more of several beneficial effects on the health of the host. By way of first non-limiting example, Bifidobacterium breve SD-B632-IT may improve the composition of the fecal microbiota in children with celiac disease, particularly by increasing the Firmicutes/Bacteroidetes ratio and/or increasing the abundance of Actinobacteria. By way of second non-limiting example, Bifidobacterium breve SD-B632-IT may modulate serum TNF-α and/or fecal short chain fatty acids. By way of third non-limiting example, Bifidobacterium breve SD-B632-IT may improve insulin sensitivity and blood glucose levels in children with obesity and insulin resistance. By way of fourth non-limiting example, Bifidobacterium breve SD-B632-IT may play a role in preventing or treating infant colic. By way of fifth non-limiting example, Bifidobacterium breve SD-B632-IT may help restore microbial balance in children with celiac disease on a gluten-free diet. By way of sixth non-limiting example, Bifidobacterium breve SD-B632-IT may support the growth of healthy gut bacteria in children with celiac disease. By way of seventh non-limiting example, Bifidobacterium breve SD-B632-IT may support healthy carbohydrate metabolism and insulin sensitivity in adolescents. By way of eighth non-limiting example, Bifidobacterium breve SD-B632-IT may improve allergic airway response in patients with asthma, seasonal allergies, or the like, and/or otherwise support healthy respiratory tract function, support healthy response to seasonal allergens, and/or improve rescue of respiratory distress during seasonal allergies.

Embodiments of the present disclosure include synbiotic compositions comprising Bifidobacterium breve SD-BR03-IT. This bacterial strain may have any one or more of several beneficial effects on the health of the host. By way of first non-limiting example, Bifidobacterium breve SD-BR03-IT may improve the host's bowel movement frequency, stool consistency, and ease of expulsion, and may alleviate symptoms of intestinal discomfort such as abdominal bloating, itching, burning, or pain. By way of second non-limiting example, Bifidobacterium breve SD-BR03-IT may improve clinical parameters of atopic dermatitis as measured by SCORAD and Dermatology Life Quality (DLQ) index, reduce microbial translocation and/or immune activation, and/or improve T-helper cell (Th) 17/regulatory T cell (T_{reg}) and Th1/Th2 ratios. By way of third non-limiting example, Bifidobacterium breve SD-BR03-IT may persist in the host's gut microbiota after cessation of administration of the synbiotic, which may rectify the dysbiotic gut microbiota of atopic dermatitis patients. By way of fourth non-limiting example, Bifidobacterium breve SD-BR03-IT may inhibit the growth of multiple E. coli biotypes, including pathogenic E. coli O157:H7. By way of fifth non-limiting example, Bifidobacterium breve SD-BR03-IT may improve the composition of the fecal microbiota in children with celiac disease, particularly by increasing the Firmicutes/Bacteroidetes ratio and/or increasing the abundance of Actinobacteria. By way of sixth non-limiting example, Bifidobacterium breve SD-BR03-IT may modulate serum TNF-α and/or fecal short chain fatty acids. By way of seventh non-limiting example, Bifidobacterium breve SD-BR03-IT may improve insulin sensitivity and blood glucose levels in children with obesity and insulin resistance. By way of eighth non-limiting example, Bifidobacterium breve SD-BR03-IT may play a role in preventing or treating infant colic.

Embodiments of the present disclosure include synbiotic compositions comprising Bifidobacterium longum SD-CECT7347-SP. This bacterial strain may have any one or more of several beneficial effects on the health of the host. By way of first non-limiting example, Bifidobacterium longum SD-CECT7347-SP may, in combination with Lacticaseibacillus casei SD-CECT9104-SP and Bifidobacterium lactis SD-CECT8145-SP, reduce SCORAD index and use of topical steroid treatments to treat flares of chronic dermatological disease. By way of second non-limiting example, Bifidobacterium longum SD-CECT7347-SP may reduce the altered expression of cellular proteins involved in disorganization of cell cytoskeleton, inflammation, and apoptosis in Caco-2 cells. By way of third non-limiting example, Bifidobacterium longum SD-CECT7347-SP may suppress the pro-inflammatory cytokine pattern and increase IL-10 production in peripheral blood mononuclear cells (PBMCs) in hosts with celiac disease. By way of fourth non-limiting example, Bifidobacterium longum SD-CECT7347-SP may influence phenotypic and functional maturation of monocyte-derived dendritic cells. By way of fifth non-limiting example, Bifidobacterium longum SD-CECT7347-SP may reduce cellular exhibition of toxic amino acid sequences and reduce expression of NF-κB, TNF-α, and IL-1β.

Embodiments of the present disclosure include synbiotic compositions comprising Lacticaseibacillus casei SD-CECT9104-SP. This bacterial strain may have any one or more of several beneficial effects on the health of the host. By way of non-limiting example, Lacticaseibacillus casei SD-CECT9104-SP may, in combination with Bifidobacterium longum SD-CECT7347-SP and Bifidobacterium lactis SD-CECT8145-SP, reduce SCORAD index and use of topical steroid treatments to treat flares of chronic dermatological disease.

Embodiments of the present disclosure include synbiotic compositions comprising Bifidobacterium lactis SD-CECT8145-SP. This bacterial strain may have any one or more of several beneficial effects on the health of the host. By way of non-limiting example, Bifidobacterium lactis SD-CECT8145-SP may, in combination with Bifidobacterium longum SD-CECT7347-SP and Lacticaseibacillus casei SD-CECT9104-SP, reduce SCORAD index and use of topical steroid treatments to treat flares of chronic dermatological disease.

Embodiments of the present disclosure include synbiotic compositions comprising Lactobacillus acidophilus SD-NCFM-US. This bacterial strain may have any one or more of several beneficial effects on the health of the host. By way of non-limiting example, Lactobacillus acidophilus SD-NCFM-US may reduce fever, rhinorrhea, cough incidence and duration, antibiotic prescriptions, and missed school days from illness in children. By way of second non-limiting example, Lactobacillus acidophilus SD-NCFM-US may decrease stool frequency and/or improve stool consistency in children with diarrhea.

Embodiments of the present disclosure include synbiotic compositions comprising Bifidobacterium animalis subsp. lactis SD-BI07-US. This bacterial strain may have any one or more of several beneficial effects on the health of the host. By way of non-limiting example, Bifidobacterium animalis subsp. lactis SD-BI07-US may reduce fever, rhinorrhea, cough incidence and duration, antibiotic prescriptions, and missed school days from illness in children. By way of second non-limiting example, Bifidobacterium animalis subsp. lactis SD-BI07-US may decrease stool frequency and/or improve stool consistency in children with diarrhea.

Aspects of the present disclosure allow for rational and systematic screening and selection of bacterial strains of interest for use in synbiotic compositions. Particularly, as described throughout this disclosure, bacterial strains of interest can be, and in preferred embodiments are, screened for a variety of functional attributes, including but by no means limited to upregulation of Nrf2 transcription factor, increased short-chain fatty acid (SCFA) production in the gut, and/or improved epithelial barrier function in the gut. As a result of this screening, individual strains or combinations of strains, each possessing one or more of these desired functional attributes and collectively possessing most or all of the desired attributes, can be included in the synbiotic composition to provide a synergistic effect on the health of the host. By way of non-limiting example, upregulation of Nrf2, increased SCFA production, and improved epithelial barrier function may, in some embodiments, mutually reinforce each other in the gut environment of the host and may therefore result in a greater improvement in overall host health than any one or two of these functional outcomes; thus, strains may be screened for these attributes and rationally selected for inclusion in the synbiotic compositions of the disclosure as a result of this screening.

### Prebiotics

It is to be expressly understood that any compound that can be converted, by a microbial strain present in the healthy human gut microbiota, into a bioactive metabolite may be provided in synbiotic compositions according to the present disclosure. By way of first non-limiting example, the prebiotic component may comprise one or more microbe-fermentable dietary fibers, i.e. polymeric or oligomeric carbohydrates, such as fructooligosaccharides (FOSs), galactooligosaccharides (GOSs), and inulin. By way of second non-limiting example, the prebiotic component may comprise one or more punicalagins, which act in the human body as powerful antioxidants and can be metabolized by certain gut bacteria into a class of dibenzopyran-6-ones known as urolithins, including but not necessarily limited to urolithin-A. By way of third non-limiting example, the prebiotic component may comprise one or more glucosinolates, which may be metabolized by the gut microbiota into isothiocyanates, which in turn have been shown to have anti-cancer properties and other beneficial roles in human health. By way of fourth non-limiting example, the prebiotic component may comprise one or more catechins, which may be metabolized by microbes in the colon into γ-valerolactones and hippuric acids, which may in turn be biotransformed in the human liver into metabolites useful for human health. By way of fifth non-limiting example, the prebiotic component may comprise one or more polyphenols, many of which are known to be metabolized by the gut microbiota into compounds that are beneficial for human health. These and other embodiments are within the scope of the present disclosure. In some embodiments, the compound may be a compound that is otherwise, from the host's perspective, biologically “inert” (i.e. can be metabolized only by one or more strains in the gut microbiota and not by the host), whereas in other embodiments the compound can be metabolizable by both the host and the gut microbiota.

### Pharmaceutical Formulations

Pharmaceutical formulations provided by the present disclosure may comprise a therapeutically effective amount of a synbiotic composition together with a suitable amount of one or more pharmaceutically acceptable vehicles so as to provide a formulation for proper administration to a patient. Suitable pharmaceutical vehicles are described in the art.

In certain embodiments, the synbiotic composition may be incorporated into pharmaceutical formulations to be administered orally. Oral administration of such pharmaceutical formulations may result in release and/or uptake of the synbiotic composition throughout the intestine. Such oral formulations may be prepared in a manner known in the pharmaceutical art and comprise the synbiotic composition and at least one pharmaceutically acceptable vehicle. Oral pharmaceutical formulations may include a therapeutically effective amount of the synbiotic composition and a suitable amount of a pharmaceutically acceptable vehicle, so as to provide an appropriate form for administration to a patient.

The synbiotic composition may be incorporated into pharmaceutical formulations to be administered by any other appropriate route of systemic administration including intramuscular, intravenous, and oral.

Embodiments of the present disclosure generally include an ingestible formulation comprising a synbiotic composition. Embodiments of the present disclosure also include methods of administration of such formulations to a subject, preferably a human and still more preferably a child, to treat a disease or condition and/or achieve a physiological objective. Particularly, in some embodiments, synbiotic compositions are co-administered, or administered in combination with, one or more other pharmaceutical formulations to achieve an adjunctive or synergistic effect, i.e. an improved effect relative to independent administration of the synbiotic composition and the one or more other pharmaceutical formulations. By way of first non-limiting example, synbiotic compositions according to the present disclosure may be co-administered with one or more pharmaceutical formulations effective to treat inflammatory bowel disease to improve the efficacy response rate of the pharmaceutical formulation in treating inflammatory bowel disease. By way of second non-limiting example, synbiotic compositions according to the present disclosure may be co-administered with one or more cancer immunotherapy drugs (e.g. a CTLA-4 antagonist and/or a PD-1 antagonist) to improve the efficacy response rate of the cancer immunotherapy drug.

Ingestible formulations of the present invention may be provided in any suitable form and physical manifestation. By way of non-limiting example, the ingestible formulation can be administered to a subject as a dietary supplement, a medicated feed, a nutraceutical composition, and a pharmaceutical composition. By way of further non-limiting example, the ingestible compositions may be provided in any suitable physical form for oral administration, such as ampules, aqueous solutions or suspensions (e.g. an infused beverage, such as an energy beverage or energy “shot”), capsules (which may or may not be chewable), drops, granules, liquids, mists, powders, sachets, syrups, tablets (e.g. chewable, saliva-soluble, and/or swallowable tablets), functionalized foods (e.g. energy or nutrition bars, cookies, gums, candies, etc.), toothpastes, sublingual articles, and the like. In some embodiments, the composition may be provided in a form, e.g. a powder, that can be applied to a food (similar to a seasoning or condiment, etc.) or mixed with a beverage. Ingestible compositions of the present invention may thus comprise any suitable pharmaceutically acceptable additives, binders, and/or fillers, and may also comprise an active pharmaceutical or therapeutic agent other than the synbiotic compositions as disclosed herein.

In embodiments, ingestible formulations of the present invention may be provided in the form of a free-flowing powder. The powder may be adapted to be mixed with, or dissolved or suspended in, a beverage (e.g., water, milk, etc.) or other liquid, which the subject then drinks or otherwise receives orally. In certain embodiments, ingestible formulations in the form of a powder may be packaged in sachets, e.g., single-use sachets comprising a single daily dose, which may be adapted to be immersed in the liquid (similar to a tea bag) to allow the ingestible formulation to diffuse into the liquid, torn open to have the contents poured into the liquid, etc. It may in some embodiments be preferable for a packaging material from which the sachet is made to have a relatively low moisture vapor transmission rate, e.g. a moisture vapor transmission rate, at 23° C. and 50% relative humidity, of less than about 0.01 grams per square meter per day, to extend the shelf life of the ingestible formulation.

Pharmaceutical formulations comprising a synbiotic composition may be manufactured by means of conventional mixing, dissolving, granulating, dragee-making, levigating, emulsifying, encapsulating, entrapping, or lyophilizing processes. Pharmaceutical formulations may be formulated in a conventional manner using one or more physiologically acceptable carriers, diluents, excipients, or auxiliaries, which facilitate processing of the synbiotic composition and one or more pharmaceutically acceptable vehicles into formulations that can be used pharmaceutically. Proper formulation is dependent upon the route of administration chosen. Pharmaceutical formulations provided by the present disclosure may take the form of sustained-release formulations suitable for administration to a patient.

Pharmaceutical formulations provided by the present disclosure may be formulated in a unit dosage form. A unit dosage form refers to a physically discrete unit suitable as a unitary dose for patients undergoing treatment, with each unit containing a predetermined quantity of the synbiotic composition calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

In certain embodiments, an oral dosage form provided by the present disclosure may be a controlled release dosage form. Controlled delivery technologies can improve the absorption of an active ingredient in a particular region or regions of the gastrointestinal tract. Controlled active ingredient delivery systems may be designed to deliver an active ingredient in such a way that the level of the active ingredient is maintained within a therapeutically effective window and effective and safe blood levels are maintained for a period as long as the system continues to deliver the active ingredient with a particular release profile in the gastrointestinal tract. Controlled active ingredient delivery may produce substantially constant blood levels of an active ingredient over a period of time as compared to fluctuations observed with immediate release dosage forms. For some applications, maintaining a constant blood and tissue concentration throughout the course of therapy is the most desirable mode of treatment. Immediate release of an active ingredient may cause blood levels to peak above the level required to elicit a desired response, which may waste the active ingredient and may cause or exacerbate toxic side effects. Controlled active ingredient delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodible matrix systems, pH independent formulations, gastric retention systems, and the like.

An appropriate oral dosage form for a particular pharmaceutical formulation provided by the present disclosure may depend, at least in part, on the gastrointestinal absorption properties of the active ingredient and/or the stability of the active ingredient in the gastrointestinal tract, the pharmacokinetics of the active ingredient and the intended therapeutic profile. An appropriate controlled release oral dosage form may be selected for a particular ingredient or combination of ingredients. For example, gastric retention oral dosage forms may be appropriate for active ingredients absorbed primarily from the upper gastrointestinal tract, and sustained release oral dosage forms may be appropriate for active ingredients absorbed primarily from the lower gastrointestinal tract. Certain active ingredients are absorbed primarily from the small intestine. In general, active ingredients traverse the length of the small intestine in about 3 to 5 hours. For active ingredients that are not easily absorbed by the small intestine or that do not dissolve readily, the window for active agent absorption in the small intestine may be too short to provide a desired therapeutic effect.

In certain embodiments, pharmaceutical formulations provided by the present disclosure may be practiced with dosage forms adapted to provide sustained release of synbiotic compositions upon oral administration. Sustained release oral dosage forms may be used to release active ingredients over a prolonged time period and are useful when it is desired that an active ingredient be delivered to the lower gastrointestinal tract, including the colon. Sustained release oral dosage forms include any oral dosage form that maintains therapeutic concentrations of an active ingredient in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

In certain embodiments, pharmaceutical compositions provided by the present disclosure may include any enteric-coated sustained release oral dosage form for administering the synbiotic composition. In one embodiment, the enteric-coated oral dosage form is administered to a patient at a dosing frequency of three times per day. In another embodiment, the enteric-coated oral dosage form is administered to a patient at a dosing frequency of twice per day. In still another embodiment, the enteric-coated oral dosage form is administered to a patient at a dosing frequency of once per day.

In certain embodiments, pharmaceutical formulations provided by the present disclosure may include any non-enteric-coated sustained release oral dosage form for administering the synbiotic composition. In one embodiment, the non-enteric-coated oral dosage form is administered to a patient at a dosing frequency of three times per day. In another embodiment, the non-enteric-coated oral dosage form is administered to a patient at a dosing frequency of twice per day. In still another embodiment, the non-enteric-coated oral dosage form is administered to a patient at a dosing frequency of once per day.

In certain embodiments, pharmaceutical formulations provided by the present disclosure may include any oral dosage form for administering the synbiotic composition. In one embodiment, the oral dosage form is administered to a patient at a dosing frequency of three times per day. In another embodiment, the oral dosage form is administered to a patient at a dosing frequency of twice per day. In still another embodiment, the oral dosage form is administered to a patient at a dosing frequency of once per day.

In certain embodiments, pharmaceutical formulations provided by the present disclosure may include any suitable dosage forms that achieve the aforementioned in vitro release profiles. Such dosage forms may be any systemic dosage forms, including sustained release enteric-coated oral dosage form and sustained release enteric-coated or non-enteric-coated oral dosage form. Examples of suitable dosage forms are described herein. Those skilled in the formulation art can develop any number of acceptable dosage forms given the dosage forms described in the examples as a starting point.

An appropriate dose of the synbiotic composition may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

The methods and formulations disclosed herein can be used to treat patients suffering from diseases, disorders, conditions, and symptoms for which synbiotic compositions are known to provide or are later found to provide therapeutic benefit. Formulations disclosed herein can be used to treat a disease chosen from adrenal leukodystrophy, AGE-induced genome damage, Alexander Disease, alopecia areata, Alper's Disease, Alzheimer's disease, amyotrophic lateral sclerosis, angina pectoris, arthritis, asthma, Balo's concentric sclerosis, Behcet's disease, bullous pemphigoid, Canavan disease, cardiac insufficiency including left ventricular insufficiency, central nervous system vasculitis, Charcot-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, herpes zoster viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, paraneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinitis pigmentosa, sarcoidosis, Schilder's Disease, subacute necrotizing myelopathy, susac syndrome, transplantation rejection, transverse myelitis, a tumor, ulcerative colitis or Zellweger's syndrome.

Methods of treating a disease in a patient provided by the present disclosure comprise administering to a patient in need of such treatment a therapeutically effective amount of a synbiotic composition of the disclosure. These methods and pharmaceutical formulations provide therapeutic or prophylactic amounts of the prebiotic compounds and/or probiotic strains following administration to a patient. The synbiotic composition may be administered in an amount and using a dosing schedule as appropriate for treatment of a particular disease.

Daily doses of compounds of the prebiotic component of the synbiotic composition may range from about 0.01 mg/kg to about 200 mg/kg, from about 0.1 mg/kg to about 175 mg/kg, from about 1 mg/kg to about 150 mg/kg, from about 5 mg/kg to about 125 mg/kg, and in certain embodiments about 100 mg/kg. In certain embodiments, compounds of the prebiotic component may be administered at a dose over time from about 1 mg to about 12 g per day, from about 10 mg to about 11 g per day, in certain embodiments from about 20 mg to about 2 g per day, in certain embodiments from about 100 mg to about 1 g per day, in certain embodiments from about 150 mg to about 650 mg per day, in certain embodiments from about 250 mg to about 550 mg per day, in certain embodiments from about 350 mg to about 450 mg per day, and in certain embodiments about 400 mg per day.

In certain embodiments, microbial strains of the probiotic component may be administered at a dose over time from about 125 million AFU to about 625 billion AFU per day, in certain embodiments from about 1.25 billion AFU to about 500 billion AFU per day, in certain embodiments from about 2.5 billion AFU to about 250 billion AFU per day, in certain embodiments from about 12.5 billion AFU to about 125 billion AFU per day, in certain embodiments from about 25 billion AFU to about 100 billion AFU per day, in certain embodiments from about 37.5 billion AFU to about 75 billion AFU per day, and in certain embodiments from about 50 billion AFU to about 62.5 billion AFU per day.

In some embodiments, the probiotic component may comprise at least about 40 million AFU per dose, and in particular embodiments about 7.5 billion AFU per dose, of Lacticaseibacillus rhamnosus SD-GG-BE. In some embodiments, the probiotic component may comprise at least about 2 billion AFU per dose, and in particular embodiments about 7.5 billion AFU per dose, of Ligilactobacillus salivarius SD-LS1-IT. In some embodiments, the probiotic component may comprise at least about 7.5 billion AFU per dose, and in particular embodiments about 7.5 billion AFU per dose, of Bifidobacterium breve SD-B632-IT. In some embodiments, the probiotic component may comprise at least about 5 billion AFU per dose, and in particular embodiments about 7.5 billion AFU per dose, of Bifidobacterium breve SD-BR03-IT. In some embodiments, the probiotic component may comprise at least about 420 million AFU per dose, and in particular embodiments about 7.5 billion AFU per dose, of Bifidobacterium longum SD-CECT7347-SP. In some embodiments, the probiotic component may comprise at least about 360 million AFU per dose, and in particular embodiments about 7.5 billion AFU per dose, of Lacticaseibacillus casei SD-CECT9104-SP. In some embodiments, the probiotic component may comprise at least about 420 million AFU per dose, and in particular embodiments about 7.5 billion AFU per dose, of Bifidobacterium lactis SD-CECT8145-SP. In some embodiments, the probiotic component may comprise at least about 7.5 billion AFU per dose, and in particular embodiments about 7.5 billion AFU per dose, of Lactobacillus acidophilus SD-NCFM-US. In some embodiments, the probiotic component may comprise at least about 7.5 billion AFU per dose, and in particular embodiments about 7.5 billion AFU per dose, of Bifidobacterium animalis subsp. lactis SD-BI07-US. In some embodiments, the absolute AFU counts of any one or more strains present in one dose of the probiotic component may vary, so long as the AFU ratio between any two or more selected microbial strains remains about equal to a predetermined ratio, e.g. a ratio as set forth above (for example, an AFU ratio between Lacticaseibacillus rhamnosus SD-GG-BE and Ligilactobacillus salivarius SD-LS1-IT of about 40 million: 2 billion, or an AFU ratio between Lactobacillus acidophilus SD-NCFM-US and Bifidobacterium lactis SD-BI07-US of about 7.5 billion: 7.5 billion).

An appropriate dose of synbiotic composition may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

The synbiotic composition may be assayed in vitro and in vivo for the desired therapeutic or prophylactic activity prior to use in humans. In vivo assays, for example using appropriate animal models, may also be used to determine whether administration of synbiotic composition is therapeutically effective.

In certain embodiments, a therapeutically effective dose of the synbiotic composition may provide therapeutic benefit without causing substantial toxicity including adverse side effects. Toxicity of the synbiotic composition and/or metabolites thereof may be determined using standard pharmaceutical procedures and may be ascertained by those skilled in the art. The dose ratio between toxic and therapeutic effect is the therapeutic index. A dose of the synbiotic composition may be within a range capable of establishing and maintaining a therapeutically effective circulating plasma and/or blood concentration of, e.g., a prebiotic ingredient that exhibits little or no toxicity.

Synbiotic composition administration may be used to treat a disease chosen from adrenal leukodystrophy, AGE-induced genome damage, Alexander Disease, alopecia areata, Alper's Disease, Alzheimer's disease, amyotrophic lateral sclerosis, angina pectoris, arthritis, asthma, Balo's concentric sclerosis, Behcet's disease, bullous pemphigoid, Canavan disease, cardiac insufficiency including left ventricular insufficiency, central nervous system vasculitis, Charcot-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, herpes zoster viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, paraneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinitis pigmentosa, sarcoidosis, Schilder's Disease, subacute necrotizing myelopathy, susac syndrome, transplantation rejection, transverse myelitis, a tumor, ulcerative colitis or Zellweger's syndrome. The underlying etiology of any of the foregoing diseases being treated may have a multiplicity of origins. Further, in certain embodiments, a therapeutically effective amount of synbiotic composition may be administered to a patient, such as a human, as a preventative measure against the foregoing diseases and disorders. Thus, a therapeutically effective amount of synbiotic composition may be administered as a preventative measure to a patient having a predisposition for and/or history of adrenal leukodystrophy, AGE-induced genome damage, Alexander Disease, alopecia areata, Alper's Disease, Alzheimer's disease, amyotrophic lateral sclerosis, angina pectoris, arthritis, asthma, Balo's concentric sclerosis, Behcet's disease, bullous pemphigoid, Canavan disease, cardiac insufficiency including left ventricular insufficiency, central nervous system vasculitis, Charcot-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, herpes zoster viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, paraneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinitis pigmentosa, sarcoidosis, Schilder's Disease, subacute necrotizing myelopathy, susac syndrome, transplantation rejection, transverse myelitis, a tumor, ulcerative colitis or Zellweger's syndrome.

In some embodiments, methods and compositions of the disclosure may be effective to improve metagenomic stability and metabolic output of the gut microbiota in subjects with irritable bowel syndrome.

In some embodiments, methods and compositions of the disclosure may be effective to improve, remediate, or restore function to the gut and airway microbiomes of subjects with mild to moderate COVID-19 symptomology.

In some embodiments, methods and compositions of the disclosure may be effective to retain or improve gut barrier integrity and/or gut microbiota composition in subjects suffering from irritable bowel syndrome (IBS). Particularly, methods and compositions of the disclosure may be effective for any one or more of (1) maintaining or increasing diversity of the gastrointestinal microbiota of subjects suffering from constipation-predominant IBS (IBS-C), (2) increasing representation of at least one of Bifidobacterium longum, Prevotella intermedia, Bacteroides helcogenes, Akkermansia muciniphila, Alistipes finegoldii, and Faecalibacterium prausnitzii and/or decreasing representation of Blautia obeum in the gastrointestinal microbiota of subjects suffering from mixed/alternating stool pattern IBS (IBS-M), (3) improving any one or more individual IBS symptoms selected from the group consisting of abdominal pain, bloating, bowel movement difficulty, and stool consistency, (4) increasing frequency of complete spontaneous bowel movements (CSBM) in subjects suffering from IBS-C, and (5) improvement in visceral sensitivity index (VSI). In some embodiments, the methods and compositions of the disclosure may be effective for any one or more of these purposes after the subject has been treated with an antibiotic, e.g. rifaximin.

In some embodiments, methods and compositions of the disclosure may be effective to ameliorate, prevent, or treat constipation, support gut barrier integrity after acute alcohol exposure, and/or support vaginal health.

The disclosure is further described by reference to the following non-limiting Examples. The Examples illustrate various aspects of the disclosure. It will be apparent to those skilled in the art that many modifications, to both materials and methods, may be practiced without departing from the scope of the disclosure.

### Example 1

### Prebiotic Utilization

Individual strains of nine probiotic bacterial strains were streak plated from frozen stock onto De Man, Rogosa and Sharpe (MRS) solid media containing 0.5 g/L L-cysteine and incubated anaerobically at 37° C. under stationary conditions for 24 hours. The nine bacterial strains were Lacticaseibacillus rhamnosus SD-GG-BE, Ligilactobacillus salivarius SD-LS1-IT, Bifidobacterium breve SD-B632-IT, Bifidobacterium breve SD-BR03-IT, Bifidobacterium longum SD-CECT7347-SP, Lacticaseibacillus casei SD-CECT9104-SP, Bifidobacterium lactis SD-CECT8145-SP, Lactobacillus acidophilus SD-NCFM-US, and Bifidobacterium animalis subsp. lactis SD-BI07-US. (Hereinafter, these nine strains may be referred to collectively as the “PDS-08 strains,” and compositions containing the nine strains may be referred to as “PDS-08 compositions” or simply “PDS-08.” The first seven listed of these strains, i.e. the nine PDS-08 strains except Lactobacillus acidophilus SD-NCFM-US and Bifidobacterium animalis subsp. lactis SD-BI07-US, may be referred to collectively as the “MXP271 strains,” and compositions containing the seven strains may be referred to as “MXP271 compositions” or simply “MXP271.”) Single colonies of each PDS-08 strain were selected and inoculated in MRS broth with 0.5 g/L L-cysteine, then anaerobically incubated overnight at 37° C. under stationary conditions to produce experimental cultures. The resulting stationary phase cultures were sub-cultured (1:100) into 96-well plates containing a medium mimicking the colonic environment that contains 5 g/L soluble starch, 2 g/L pectin, 1 g/L guar gum, 4 g/L mucin, 2 g/L xylan, 2 g/L arabinogalactan, 3 g/L casein, 5 mL/L 0.15% (w/v) peptone water, 5 g/L tryptone, 0.4 g/L bile salts, 4.5 g/L yeast extract, 0.005 g/L FeSO₄·7H₂O, 4.5 g/L NaCl, 4.5 g/L KCl, 0.5 g/L KH₂PO₄, 1.25 g/L MgSO₄·7H₂O, 0.15 g/L CaCl₂·6H₂O, 1.5 g/L NaHCO₃, 0.8 g/L L-cysteine, 0.05 g/L hemin, and 1 mL/L Tween 80. Some samples were spiked with 1 wt % of one of three commercially available prebiotic compositions—Orafti® Synergy 1 (approximately 50 wt % inulin, 50% fructooligosaccharides), Glucidex® (maltodextrin), and waxy maize (complex starch mixture with high amylopectin content)—while other samples were not spiked to serve as controls. The plates were incubated for 24 hours, during which time the optical density at 600 nanometers (OD₆₀₀) was measured every 30 minutes using a microplate reader. Three to nine biological replicates with technical triplicates were performed.

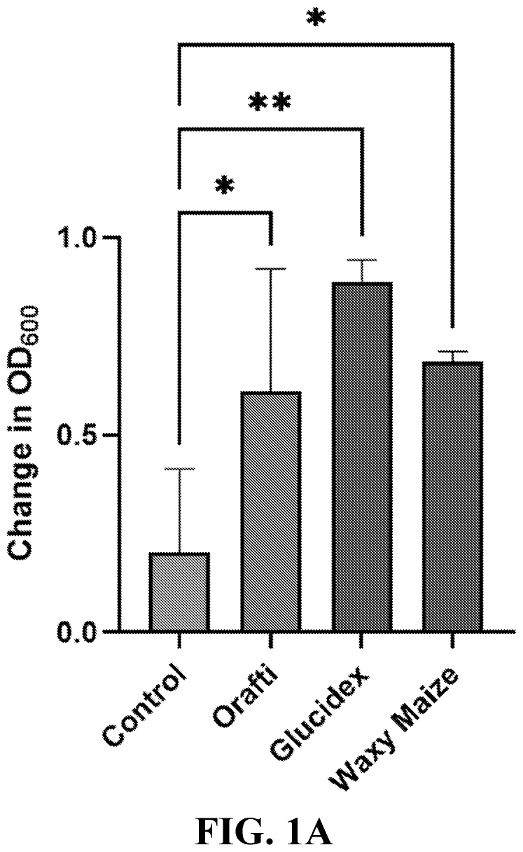

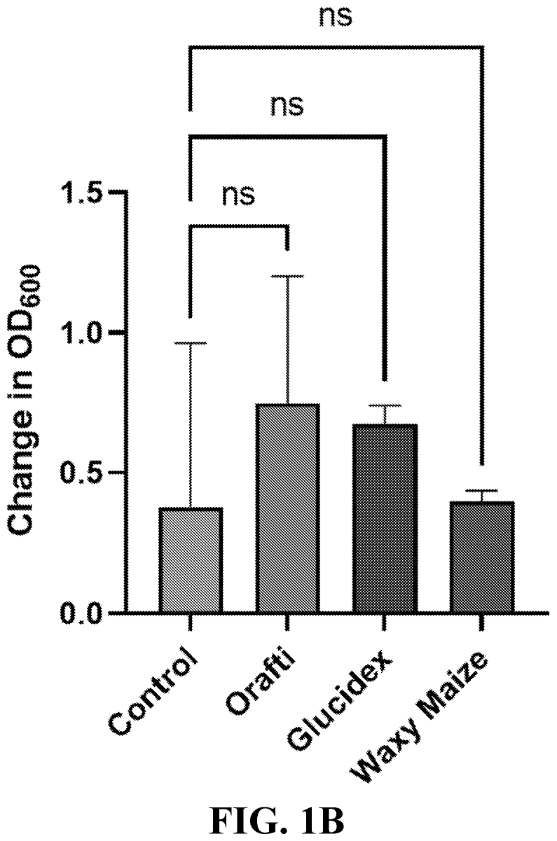

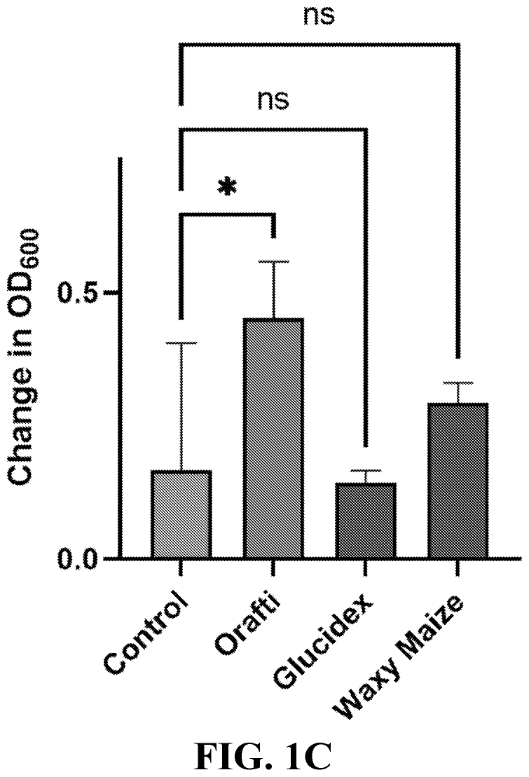

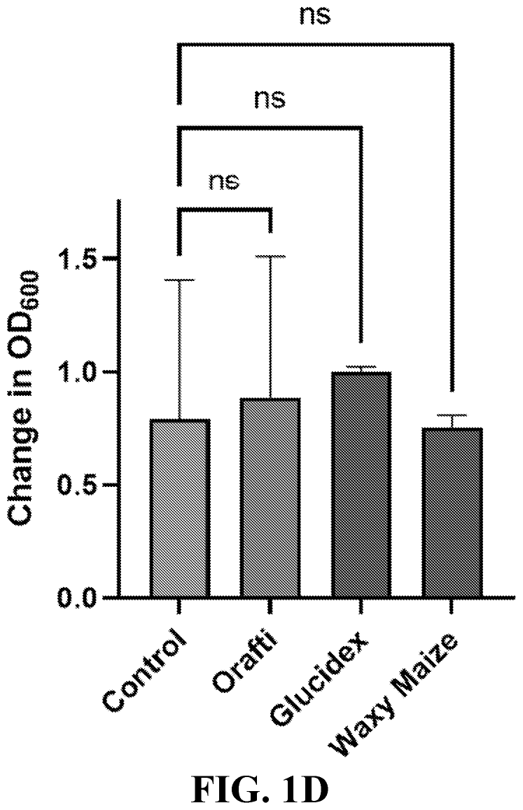

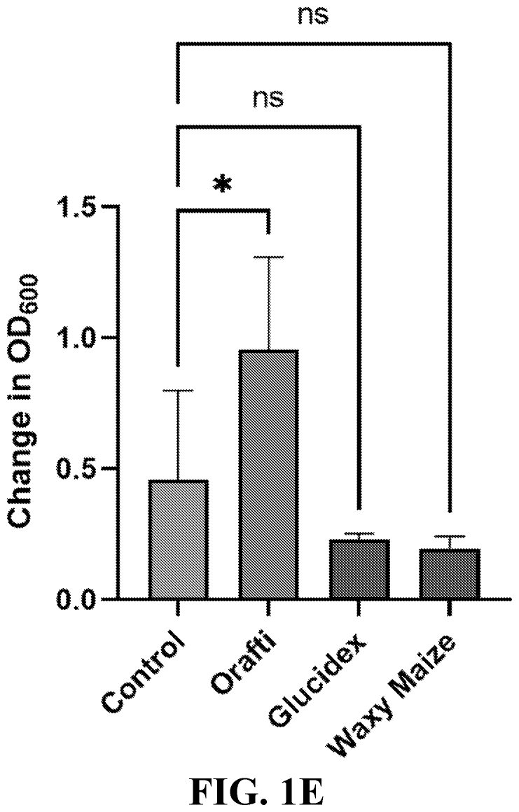

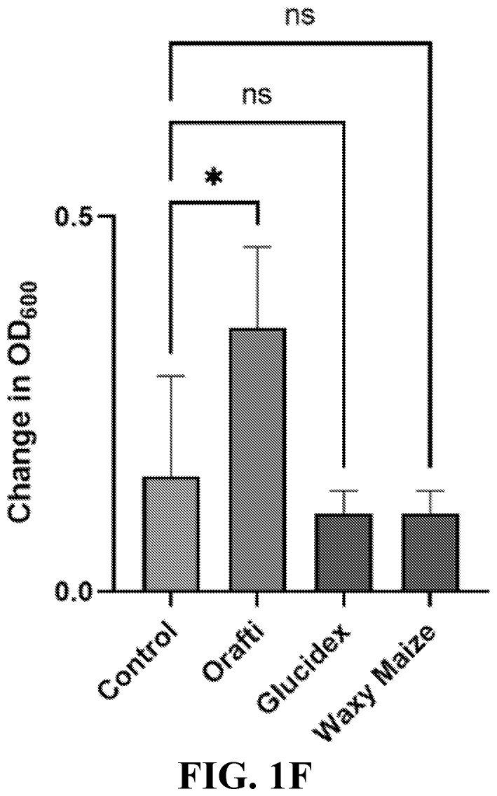

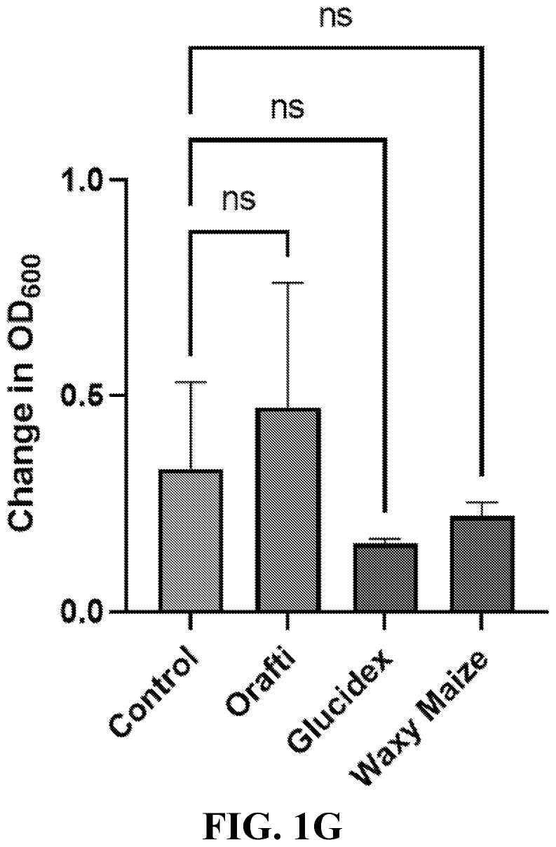

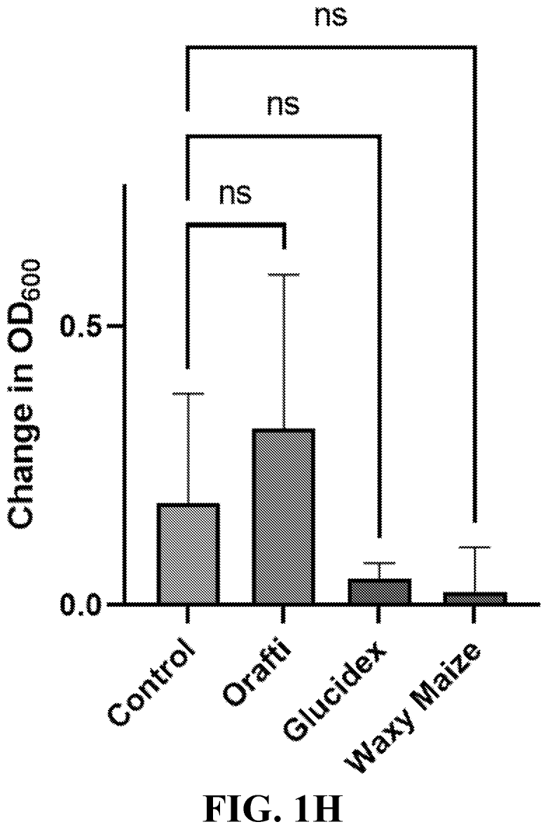

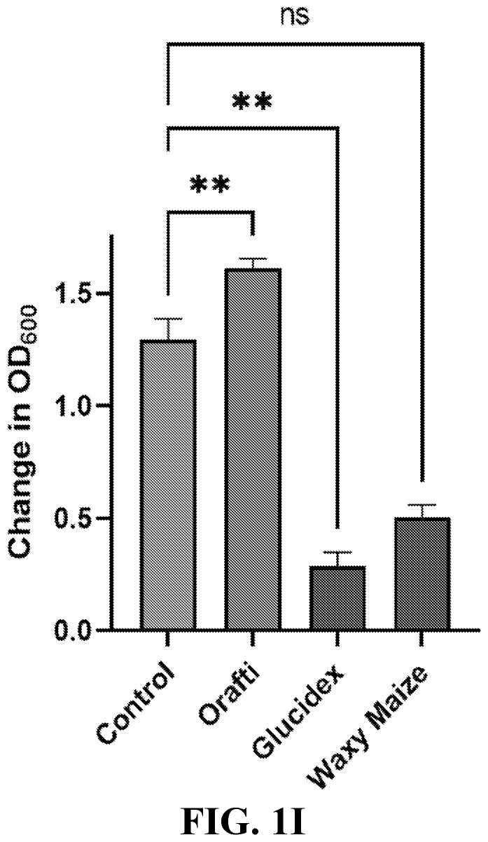

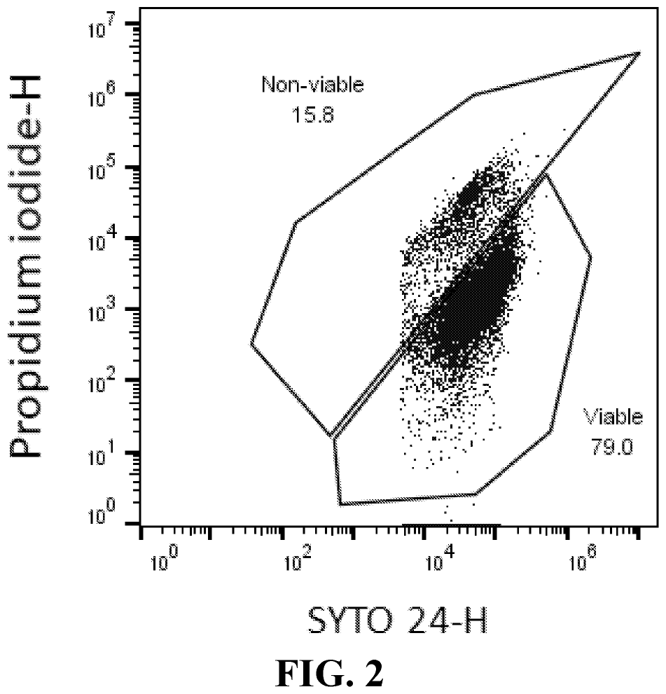

FIG. 1A illustrates the growth of Lacticaseibacillus casei SD-CECT9104-SP, FIG. 1B illustrates the growth of Lacticaseibacillus rhamnosus SD-GG-BE, FIG. 2C illustrates the growth of Bifidobacterium breve SD-BR03-IT, FIG. 1D illustrates the growth of Bifidobacterium lactis SD-CECT8145-SP, FIG. 1E illustrates the growth of Ligilactobacillus salivarius SD-LS1-IT, FIG. 1F illustrates the growth of Bifidobacterium longum SD-CECT7347-SP, FIG. 1G illustrates the growth of Lactobacillus acidophilus SD-NCFM-US, FIG. 1H illustrates the growth of Bifidobacterium animalis subsp. lactis SD-Bi-07-US, and FIG. 1I illustrates the growth of Bifidobacterium breve SD-B632-IT. Comparison of the changes in OD₆₀₀ reveals that the Orafti® prebiotic composition significantly enhanced the growth of Lacticaseibacillus casei SD-CECT9104-SP (FIG. 1A), Bifidobacterium breve SD-BR03-IT (FIG. 1C), Ligilactobacillus salivarius SD-LS1-IT (FIG. 1E), Bifidobacterium longum SD-CECT7347-SP (FIG. 1F), and Bifidobacterium breve SD-B632-IT (FIG. 1I) relative to the control group. For the other four tested strains, a trend of increased growth in the presence of the Orafti® composition compared to the control group is observed, although this trend is not statistically significant. Notably, the Orafti® composition better stimulated the growth of all strains, except Lacticaseibacillus casei SD-CECT9104-SP, than both the Glucidex® and waxy maize compositions; in fact, in the case of Bifidobacterium animalis subsp. lactis SD-Bi-07-US (FIG. 1H) specifically, the Glucidex® and waxy maize compositions appear to inhibit growth. (Significance was determined by comparing the change in OD₆₀₀ (FIGS. 1A through 1I) against each test substrate with a one-way ANOVA (parametric data) or Kruskal-Wallis (non-parametric data) test paired with a Dunnett's multiple comparison; results are marked in FIGS. 1A through II as “ns” if not significant, “*” if significant to P<0.05, “**” if significant to P<0.01, “***” if significant to P<0.001, and “****” if significant to P<0.0001.) Taken together, these data indicate significant growth enhancement for at least five of the PDS-08 strains by the Orafti® prebiotic composition, while growth of the other four strains was not negatively impacted. This provides a rationale for the employment of an inulin/fructooligosaccharide prebiotic composition, e.g. Orafti® Synergy 1, in formulation of PDS-08 synbiotic compositions.

### Example 2

### Microbial Viability in Synbiotic Product

A synbiotic composition comprising 6.3 grams per dose of Orafti® Synergy 1 (the prebiotic component) and a combined potency of 5.75·10¹⁰ AFU per dose of the PDS-08 microbial strains (the probiotic component) was formulated. The synbiotic composition was formulated as a free-flowing powder in which the only ingredients were the prebiotic and probiotic components. After formulation of the synbiotic composition, a sample of the probiotic component was obtained and quantification of total, viable, and dead bacterial quantification was carried out by the flow cytometry analysis technique of fluorescence-activated cell sorting (FACS). Specifically, a 10-fold serial dilution of the sample was double-stained with 0.01 mM SYTO™ 24 green fluorescent nucleic acid stain and 3 μM propidium iodide for 15 minutes at 37° C. under dark, anaerobic conditions. Samples were then analyzed on a BD FACSVerse machine using the high-flowrate setting, and bacteria were separated from medium debris and signal noise by applying a threshold level of 200 on the SYTO channel. Flow cytometry data were analyzed using FlowJo version 10.5.2 and reported as total bacterial counts in log units.

Referring now to FIG. 2, FACS analysis revealed that 79.0% of cells in the formulated synbiotic composition fell within the viable fraction, whereas 15.8% appeared dead and the remaining minor fraction (approximately 5.2%) were identified as viable but non-culturable. This high survivability justifies classification of the PDS-08 microbial component of the composition as a true probiotic, and thus the composition as a whole can conceptually be classified as a true synbiotic.

### Example 3

### Survival of Probiotic Strains Along Upper Gastrointestinal Tract

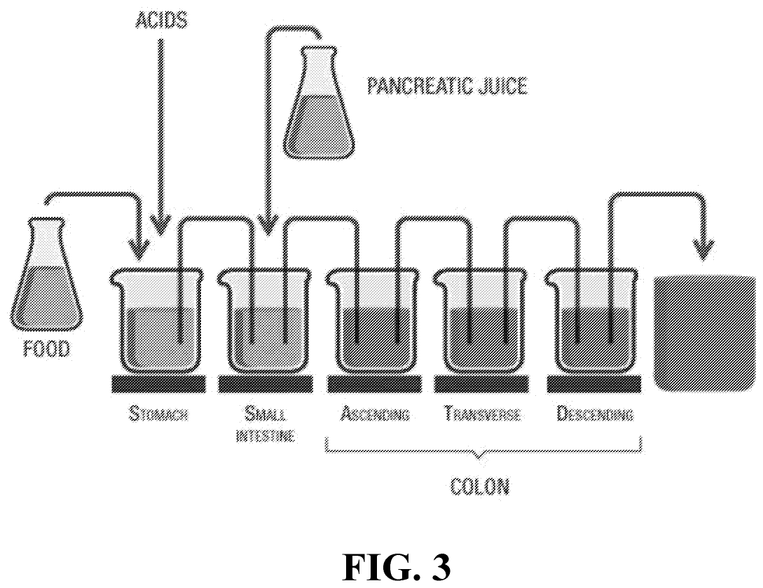

Synbiotic compositions may be tested to confirm the survival of the microbial consortia of the probiotic component using a simulator of the human intestinal microbial ecosystem (SHIME) 400, illustrated in FIG. 3. Particularly, the SHIME 400 includes a stomach vessel 410, a small intestine vessel 420, an ascending colon vessel 430, a transverse colon vessel 440, and a descending colon vessel 450. The stomach vessel 410 receives and mixes the test composition 401 and gastric acids 402 via pumps, as well as a supply of nitrogen gas. Output from the stomach vessel 410 is pumped to the small intestine vessel 420, where it is mixed with pancreatic juice 411 received via a pump. Output from the small intestine vessel 420 is pumped to the ascending colon vessel 430, which is pH-controlled to simulate the human ascending colon. Output from the ascending colon vessel 430 is pumped to the transverse colon vessel 440, which is pH-controlled to simulate the human transverse colon. Output from the transverse colon vessel 440 is pumped to the descending colon vessel 450, which is pH-controlled to simulate the human descending colon. Output from the descending colon vessel 450 is finally pumped to an effluent tank 451. The SHIME 400 thus recreates the physiological and biological conditions (e.g. food uptake, peristalsis, digestive enzymes, pancreatic and bile acids, residence time, etc.) representative of the human gastrointestinal tract. (Further details regarding the SHIME model can be found in K. Molly et al., “Development of a 5-step multi-chamber reactor as a simulation of the human intestinal microbial ecosystem,” 39 Applied Microbiology and Biotechnology 254 (May 1993), the entirety of which is incorporated herein by reference.)

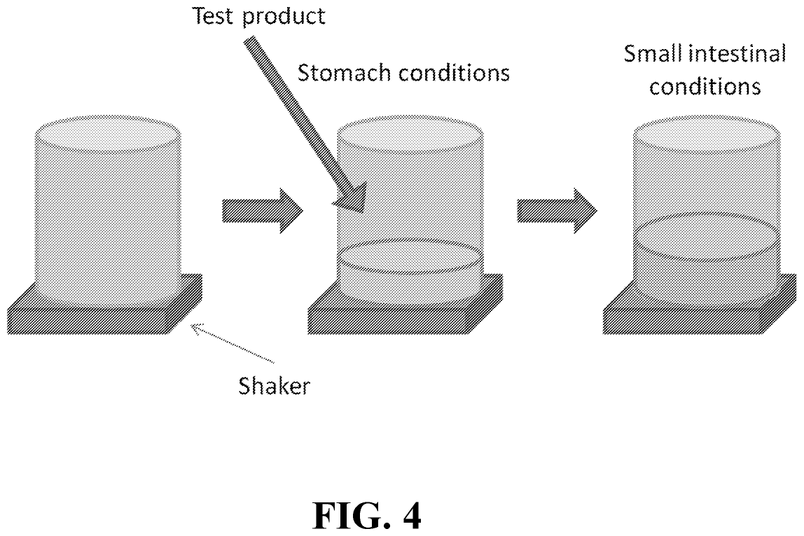

Referring now to FIG. 4, an adapted SHIME system 500 was used to study survival of probiotic strains in the upper gastrointestinal tract. The adapted SHIME system 500 comprises a single reactor 505 and a shaker 560 and uses these components to represent the physiological conditions of the stomach and small intestine within a single reactor 505 over time. To mimic fed or fasted conditions, a specific gastric suspension including a test composition 501 is added to the reactor 505 and the reactor 505 is agitated using the shaker 560 to simulate a stomach condition 510. Subsequently, a standardized enzyme and bile liquid is added to the reactor 505 and the reactor 505 is agitated using the shaker 560 to simulate a small intestine condition 520. Incubation conditions are optimized to resemble in vivo conditions in the different regions of the gastrointestinal tract for fasted or fed conditions.

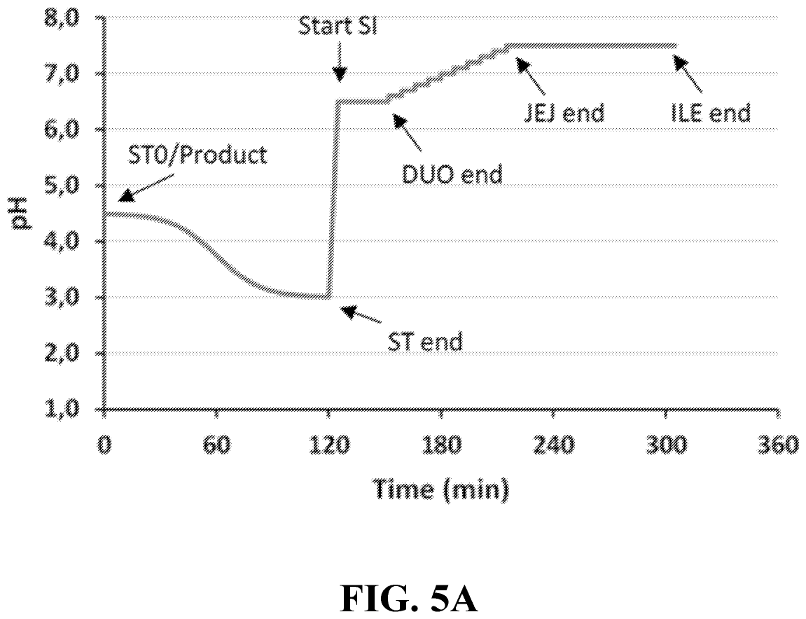

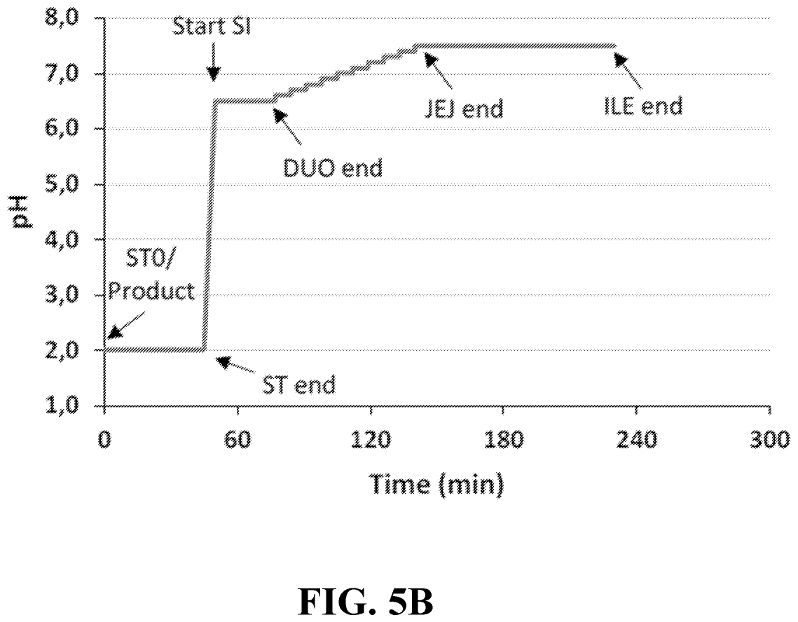

To simulate a stomach condition 510 in a fed state, the gastric suspension comprises, in addition to the test composition 501, pepsin (supplied with activity being standardized by measuring absorbance increase at 280 nanometers of trichloroacetic acid-soluble products upon digestion of a hemoglobin reference protein), phosphatidylcholine, arabinogalactan, pectin, xylan, starch, glucose, yeast extract, peptone, mucins, and L-cysteine hydrochloride, with further levels of salts (sodium chloride and potassium chloride) as recommended in Alan Mackie and Neil Rigby, “InfoGest Consensus Method,” The Impact of Food Bioactives on Health 13 (2015) (hereinafter “Mackie,” the entirety of which is incorporated herein by reference). The gastric suspension is incubated in the reactor 505 for 2 hours at 37° C., while mixing via stirring using the shaker 560, with sigmoidal decrease of the pH profile from 4.6 to 3.0 as illustrated in FIG. 5A. Samples of the gastric suspension are taken at the beginning (t=0, “STO/Product” in FIG. 5A) and end (t=120 minutes, “ST end” in FIG. 5A) of the incubation period.

To simulate a stomach condition 510 in a fasted state, the gastric suspension comprises, in addition to the test composition 501, pepsin and phosphatidylcholine in amounts equal to one-fourth the amounts used in the fed-state suspension, plus the mucins and salts of the fed-state suspension. The gastric suspension is incubated in the reactor 505 for 45 minutes at 37° C., while mixing via stirring using the shaker 560, at a constant pH of 2.0 as illustrated in FIG. 5B. Samples of the gastric suspension are taken at the beginning (t=0, “STO/Product” in FIG. 5B) and end (t=45 minutes, “ST end” in FIG. 5B) of the incubation period.

To simulate a small intestinal condition 520 in a fed state, a raw animal pancreatic extract (pancreatin) containing enzymes as described by Mackie is added to the reactor 505, as is 10 mM bovine bile extract (which more closely approximates human bile, in terms of taurocholate and glycocholate content, than does porcine bile). The pH is rapidly increased to 6.5 and kept constant at 6.5 for 27 minutes (the “duodenal phase”), then increased stepwise to 7.5 over 63 minutes (the “jejunal phase”), and finally kept constant at 7.5 for 90 minutes (the “ileal phase”). Throughout the small intestinal condition 520, the temperature is maintained at 37° C. and the suspension is mixed via stirring using the shaker 560. Samples are taken at the end of each of the duodenal, jejunal, and ileal phases (i.e. t=147 minutes/“DUO end,” t=210 minutes/“JEJ end,” and t=300 minutes/“ILE end,” respectively, in FIG. 5A).

To simulate a small intestinal condition 520 in a fasted state, pancreatin is added in an amount equal to one-fifth the amount used in the fed state and bile salts are added in an amount equal to one-third the amount used in the fed state (i.e. 3.33 mM bovine bile extract). The pH is rapidly increased to 6.5 and kept constant at 6.5 for 27 minutes (the “duodenal phase”), then increased stepwise to 7.5 over 63 minutes (the “jejunal phase”), and finally kept constant at 7.5 for 90 minutes (the “ileal phase”). Throughout the small intestinal condition 520, the temperature is maintained at 37° C. and the suspension is mixed via stirring using the shaker 560. Samples are taken at the end of each of the duodenal, jejunal, and ileal phases (i.e. t=72 minutes/“DUO end,” t=135 minutes/“JEJ end,” and t=225 minutes/“ILE end,” respectively, in FIG. 5B).

From each sample, the number of viable microbial cells was determined by the flow cytometry analysis technique of fluorescence-activated cell sorting (FACS). Specifically, a ten-fold dilution series of each sample was prepared in anaerobic phosphate-buffered saline (PBS). Assessment of the viable population in each sample was done by staining the dilutions with SYTO™ 24 stain and propidium iodide. Samples were analyzed on a BD Accuri C6 Plus flow cytometer using the high flow rate. Bacterial cells were separated from medium debris and signal noise by applying two threshold values: a primary forward scatter height (FSC-H) threshold of 500 and a secondary FL-1 threshold of 700. Samples were analyzed in triplicate and the results are reported as the average logarithm of counts per reactor ±1 standard deviation.

For each of the fed and fasted states, triplicates of two different synbiotic compositions (referred to hereinafter as the “naked strain” composition and the “microencapsulated strain” composition) were tested as the test composition 501; a single “blank” control was also tested. The naked strain composition consisted, on a per-reactor basis, of 3.0·10¹⁰ AFU of non-microencapsulated MXP271 bacterial strains, 5.0·10⁹ AFU of non-microencapsulated Bifidobacterium animalis subsp. lactis SD-BI07-US, 3.0·10¹⁰ AFU of non-microencapsulated Lactobacillus acidophilus SD-NCFM-US, and 6.025 g of Orafti® Synergy 1 prebiotic. The microencapsulated strain composition consisted, on a per-reactor basis, 3.0·10¹⁰ AFU of MXP271 bacterial strains encapsulated in a vegetable oil and lipid matrix, 5.0·10⁹ AFU of non-microencapsulated Bifidobacterium animalis subsp. lactis SD-BI07-US, 3.0·10¹⁰ AFU of non-microencapsulated Lactobacillus acidophilus SD-NCFM-US, and 6.025 g of Orafti® Synergy 1 prebiotic.

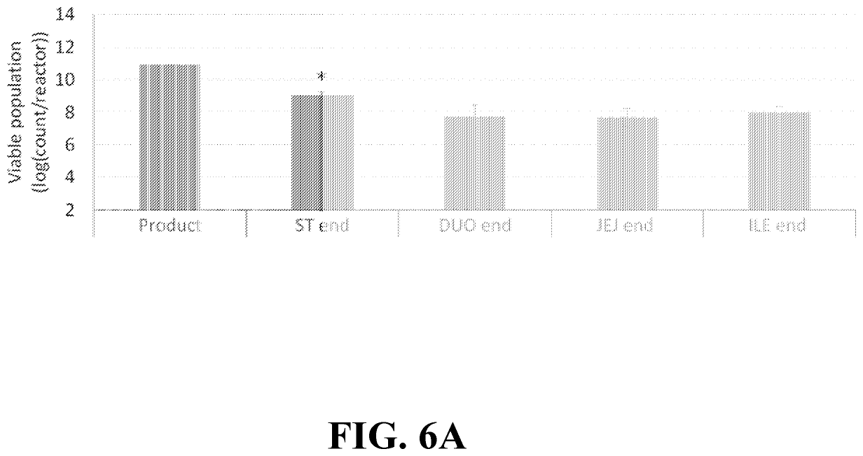

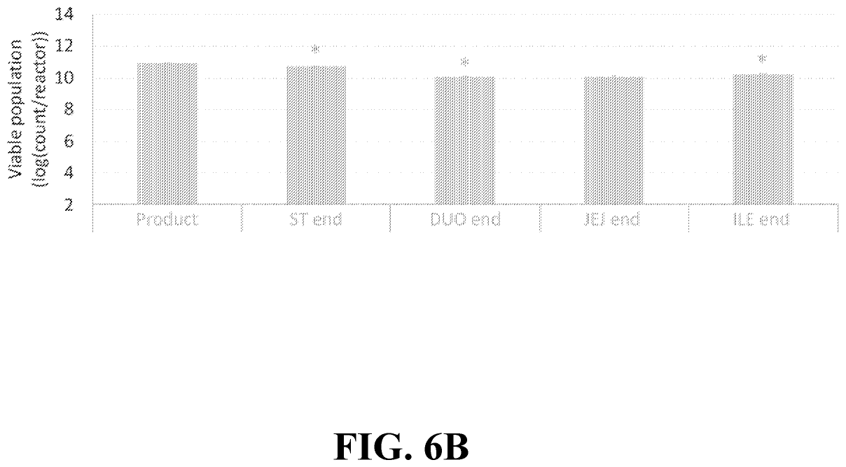

Referring now to FIGS. 6A and 6B, it was observed that the viability of the strains in the naked strain compositions was negatively affected by stomach and small intestinal conditions in both the fasted and fed states. However, the reduction in viability was more pronounced in the fasted state; without wishing to be bound by any particular theory, the present inventors hypothesize that this difference was caused by the difference in pH profile (i.e. the lower-pH stomach conditions in the fasted state than in the fed state). The viability reduction continued during the duodenal phase, although this reduction was not statistically significant in the fasted state. The bile salts added at the beginning of the duodenal phase can be highly toxic to bacterial cells, especially at relatively low pH. As pH increased in the jejunal and ileal phases, no further reduction in viability was observed, and in fact the contrary (i.e. an increase in cell viability) was observed during the ileal phase in both the fasted state and the fed state, though this increase was statistically significant only in the fed state. By the end of the ileal phase, conditions in the fasted state resulted in a reduction in viable population density of 99.85% (survival of 0.15%), from 10^{10.91}=8.15·10¹⁰ AFU/reactor at the beginning of the stomach incubation to 10^{7.97}=1.20·10⁸ AFU/reactor at the end of the ileal phase, while conditions in the fed state resulted in a reduction in viable population density of 80.67% (survival of 19.33%), from 10^{10.91}=8.15·10¹⁰ AFU/reactor at the beginning of the stomach incubation to 10^{10.19}=1.57·10¹⁰ AFU/reactor at the end of the ileal phase. (In FIGS. 6A and 6B, statistically significant reductions in viability from one phase to another are marked with asterisks. Statistical significance was determined by Student's t-test with a confidence interval of 95%.)

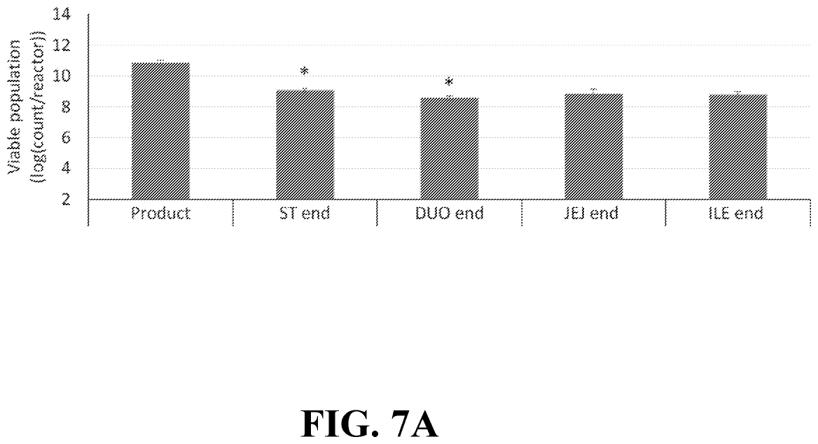

Referring now to FIGS. 7A and 7B, it was again observed that the viability of the strains in the microencapsulated strain compositions was negatively affected by stomach and small intestinal conditions in the fasted state. In the fed state, on the other hand, the viability remained largely unaffected; without being bound by any particular theory, the present inventors again attribute this difference between the fasted and fed states to the difference in pH profile during stomach incubation. During the duodenal phase, both the fasted and fed states resulted in a significant reduction of the viable population density; without wishing to be bound by any particular theory, the present inventors again attribute this to a toxic or other negative effect of the bile salts that are added at the beginning of the duodenal phase. The reduction in viable population density in the fasted state was, however, less pronounced for the microencapsulated strain formulation (FIG. 7A) than for the naked strain formulation (FIG. 6A); without wishing to be bound by any particular theory, the present inventors hypothesize that this difference is the result of more delayed release of the microencapsulated strains as compared to the naked strains, which may have a protective effect during incubation in the stomach and at least a part of the duodenal phase. After the duodenal phase, no further reduction in viable population was observed, and, as for the naked strain compositions, an increase in viability was even observed during the ileal phase in the fed state. By the end of the ileal phase, conditions in the fasted state resulted in a reduction in viable population density of 99.12% (survival of 0.88%), from 10^{10.87}=7.61·10¹⁰ AFU/reactor at the beginning of the stomach incubation to 10^{8.80}=6.70·10⁸ AFU/reactor at the end of the ileal phase, while conditions in the fed state resulted in a reduction in viable population density of 64.27% (survival of 35.73%), from 10^{10.87}=7.61·10¹⁰ AFU/reactor at the beginning of the stomach incubation to 10^{10.43}=2.72·10¹⁰ AFU/reactor at the end of the ileal phase. (In FIGS. 7A and 7B, statistically significant reductions in viability from one phase to another are marked with asterisks. Statistical significance was determined by Student's t-test with a confidence interval of 95%.)

It can be concluded that administration of microencapsulated strain compositions may generally be preferable, in terms of probiotic organism survival, to administration of naked strain compositions. Without wishing to be bound by any particular theory, the present inventors hypothesize that this may indicate that microencapsulation provides a protective effect on the PDS-08 probiotic strains during transit through the stomach and small intestine. In addition, administration of synbiotic compositions under fed conditions resulted in higher survival relative to administration under fasted conditions, indicating that administration in a fed state (e.g. when a subject has recently eaten) may be a recommended approach for delivering the greatest number of viable bacterial cells to the site of probiotic activity (i.e. the distal ileum and colon).

### Example 4

### Short-Term Colonic Batch Incubation

Subsequent to the experiments of Example 3, the predigested upper GIT suspensions subjected to treatment in the adapted SHIME system 500 were used in short-term single-stage colonic incubations. In these experiments, a representative dose of the predigested upper GIT suspension was combined with a representative bacterial inoculum under conditions characteristic of the proximal large intestine. This bacterial inoculum can be derived from an already “in vitro adapted” microbial community (e.g. from the ascending colon vessel 430 of a SHIME system 400) or from a fresh or cryopreserved fecal sample. In this Example, two types of colonic incubation were conducted: non-sterile incubations, in which the bacterial inoculum was derived from a fresh fecal sample of a child aged 6-10 years and added directly to the colonic reactor, and sterile incubations, in which the bacterial inoculum was first filter-sterilized to remove the bacterial background. In the sterile incubations, the use of the filter-sterilized inoculum allows for recreation of the metabolic background encountered by probiotic strains in the colon.

At the start of the short-term colonic incubations, a predigested upper GIT suspension (taken as an end product from the experiments of Example 3) and a fresh or filter-sterilized bacterial inoculum were added to a SHIME nutritional medium containing basal nutrients that are present in the colon (9.27 g/L K₂HPO₄, 29.06 g/L KH₂PO₄, 3.57 g/L NaHCO₃, 3.57 g/L yeast extract, 3.57 g/L peptone, 1.78 g/L mucin, 0.89 g/L L-cysteine-HCl, and 3.57 mL/L Tween 80). Orafti® Synergy 1 prebiotic was added to each colonic reactor at a concentration of 4.3 g/L, and in each case a volume of predigested suspension was selected to achieve a total prebiotic concentration in each reactor of no more than 5 g/L (the maximum concentration that can be tested in short-term colonic incubation experiments; higher concentrations result in excessive acidification). Incubations were performed over the course of 48 hours at a temperature of 37° C. under anaerobic conditions with shaking (90 rpm). The below Table 1 sets out the details for each of six different experimental setups, each of which was performed in triplicate (18 replicates total). Samples were collected for analysis after 0, 1, 24, and 48 hours of incubation.

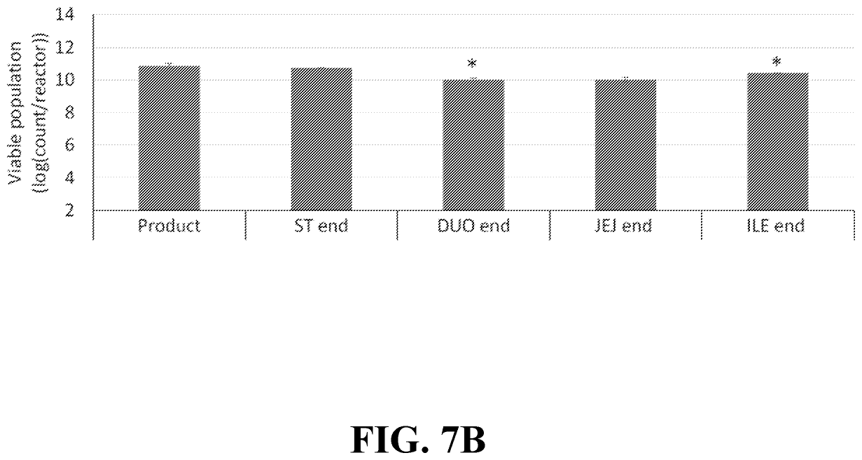

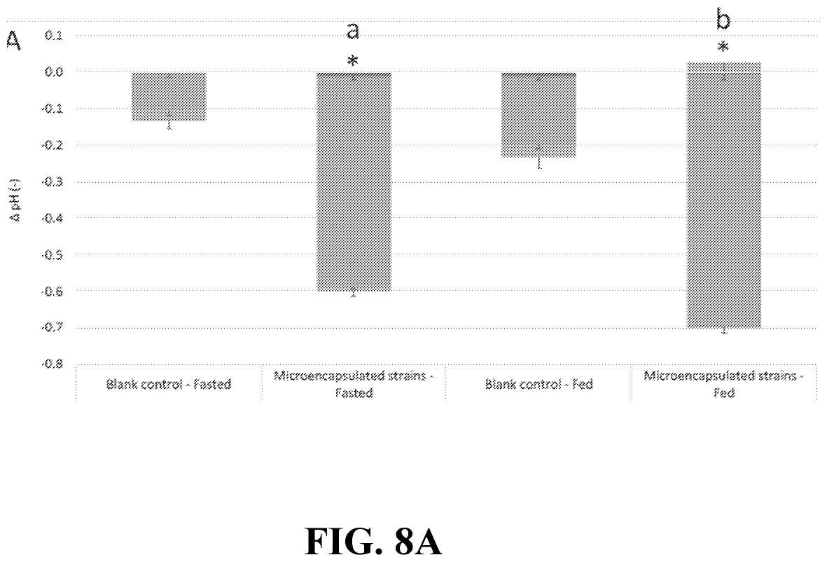

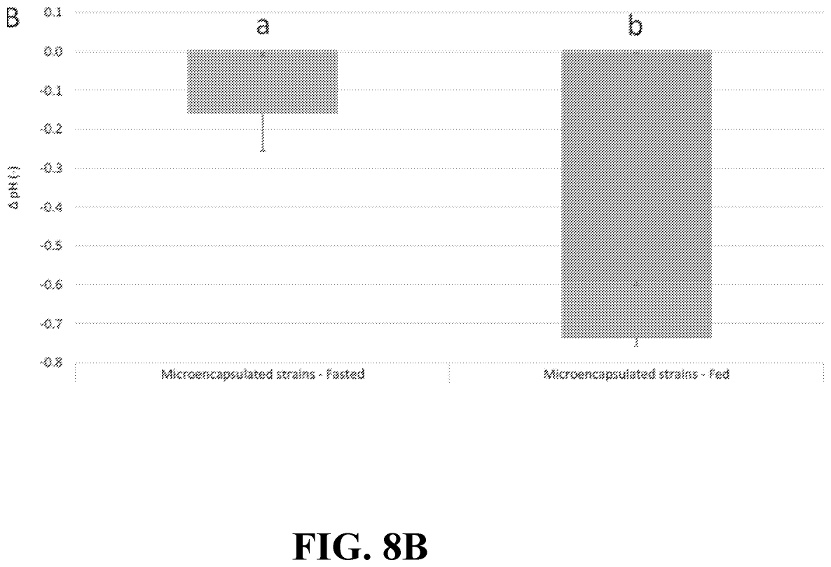

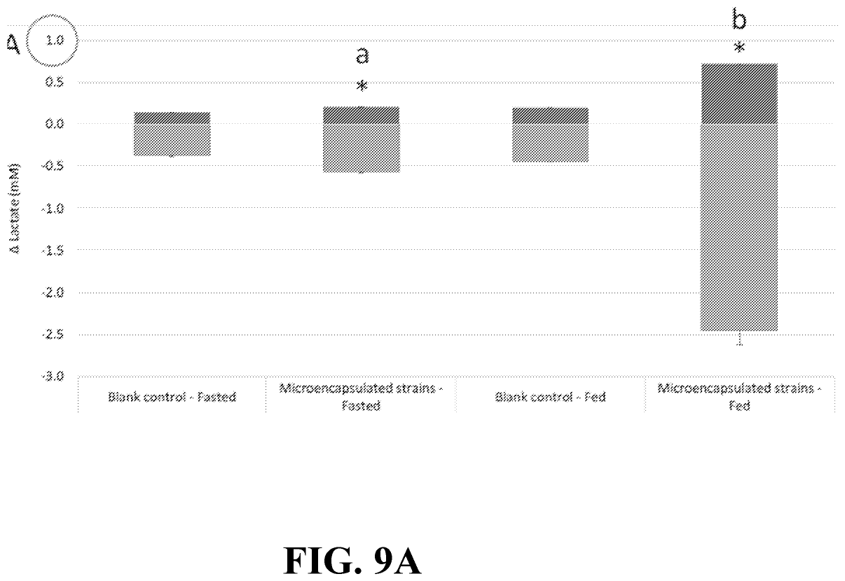

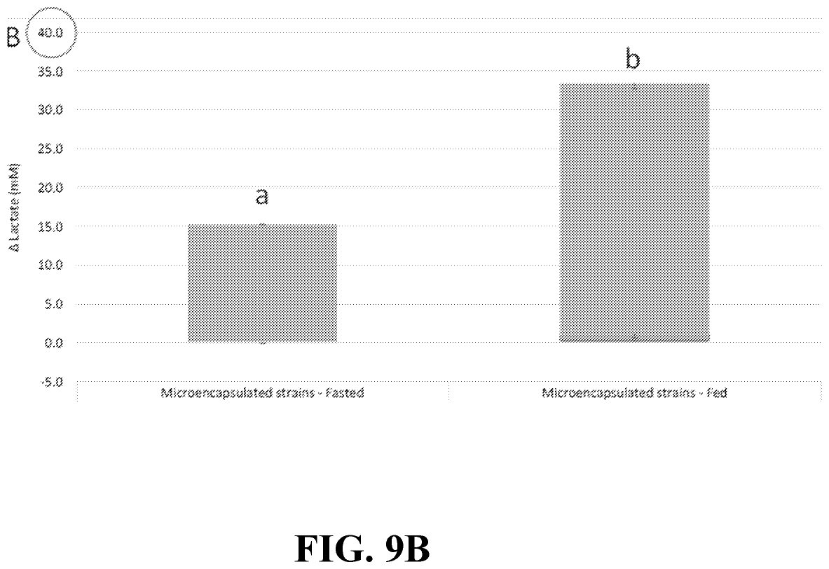

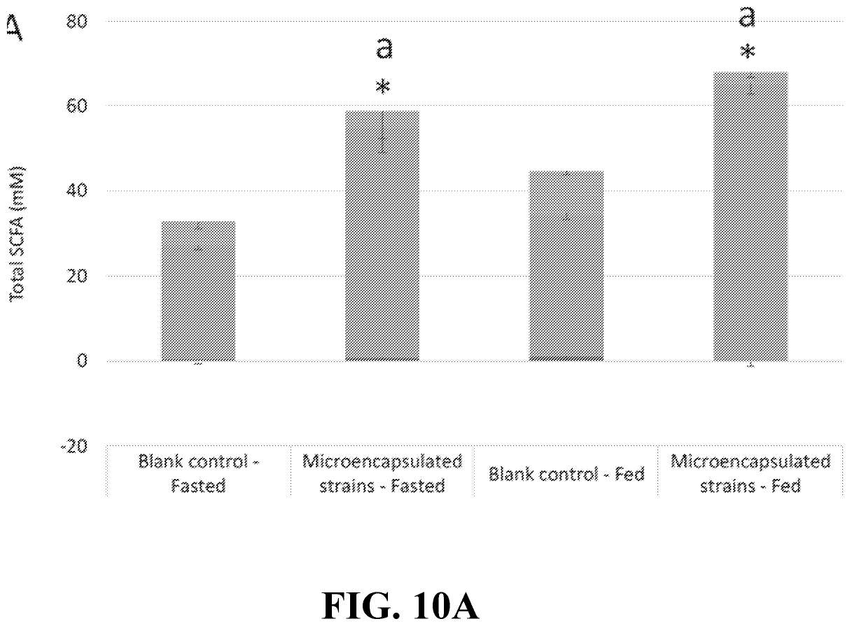

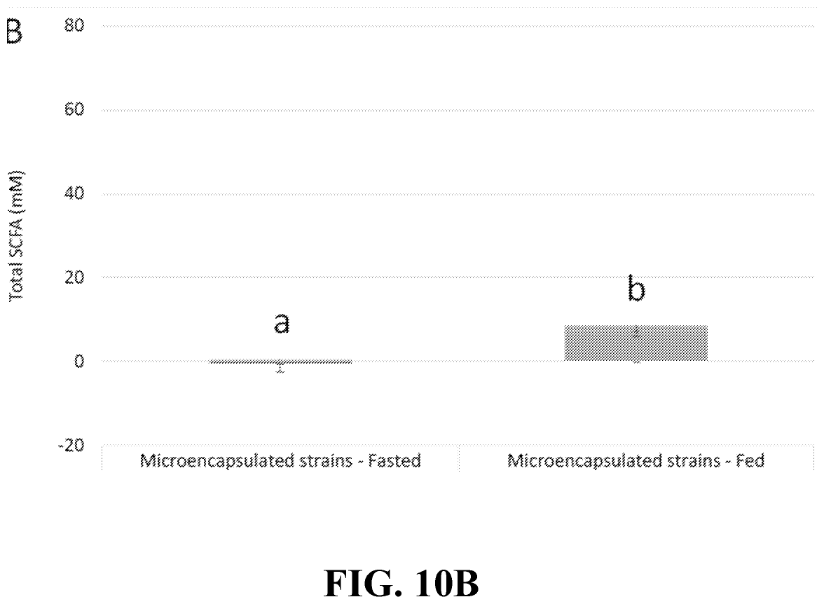

In FIGS. 8A through 14B, reported statistics are averages of the three replicates for each experimental setup. Statistically significant differences with respect to pH change (FIGS. 8A, 8B), lactate production (FIGS. 9A, 9B), and total SCFA production (FIGS. 10A, 10B) are determined over the course of the entire 48-hour incubation period. For both sterile and non-sterile incubations, significant differences between the fed and fasted test products with respect to each other are indicated by different lowercase letters (e.g. “a” vs. “b”). For non-sterile incubations only, significant differences between a microencapsulated strain test product (whether under fed or fasted conditions) and the corresponding blank/control are indicated by an asterisk. All tests for statistical significance were carried out using Student's t-test with a confidence interval of 95%.

TABLE 1

| Vessel nos. | Upper GIT suspension | Inoculum |
|---|---|---|
| 1, 2, 3 | Blank/control, fed | Fresh |
| 4, 5, 6 | Blank/control, fasted | Fresh |
| 7, 8, 9 | Microencapsulated, fed | Fresh |
| 10, 11, 12 | Microencapsulated, fed | Filter-sterilized |
| 13, 14, 15 | Microencapsulated, fasted | Fresh |
| 16, 17, 18 | Microencapsulated, fasted | Filter-sterilized |

In the colonic incubation experiments of this Example, three relevant markers of microbial metabolic activity were recorded: change in pH, short-chain fatty acid (SCFA) production, and change in lactate concentration. The degree of acidification during the experiment is a measure of the intensity of bacterial metabolism (fermentation) at any given point during incubation and therefore gives an indication of the speed of metabolism. SCFA production is an assessment of the microbial metabolism of carbohydrates (to produce acetate, propionate, and butyrate) or proteins (to produce branched SCFAs) and can be compared to typical fermentation patterns for normal gastrointestinal microbiota. Finally, as the intestine harbors both lactate-producing bacteria (e.g. lactic acid bacteria) and lactate-consuming bacteria, changes in lactate concentration can be indicative of the balance between these organisms. Lactate also decreases the pH of the environment, acts as an antimicrobial agent, and can be rapidly converted to other simple SCFAs (e.g. acetate, butyrate, propionate) by other microorganisms.

### pH Change

Monitoring the pH during short-term colonic incubation provides a reliable indication of the production of SCFAs, lactate, and ammonium (NH₄⁺). Generally, a pH drop is observed during the first 24 hours of incubation due to the formation of SCFAs and/or lactate. This pH drop is often followed by a pH increased during the second 24 hours of incubation due to proteolytic fermentation, which results in the production of, inter alia, NH₄⁺, and due to conversion of stronger acids into weaker acids (for instance, lactate to propionate and/or butyrate) through cross-feeding.

Referring now to FIGS. 8A and 8B, for the blank/control suspensions, a reduction of the pH over the 48-hour incubation period was observed under both fasted and fed conditions, but this reduction was more pronounced under fed conditions (FIG. 8A, third column, ΔpH=−0.23) than under fasted conditions (FIG. 8A, first column, ΔpH=−0.13); without wishing to be bound by any particular theory, the present inventors hypothesize that this can be explained by the higher nutrient availability, originating from the difference in gastric juice composition, under fed conditions. The pH decrease was especially marked between 1 and 24 hours of incubation, indicating that fermentation of the available substrates occurred mainly during this time interval. Overall, the present inventors conclude that the gut microbial community of the pediatric fecal donor was able to ferment the available substrates in the colonic medium and predigested upper GIT suspension.

For non-sterile incubations of the predigested microencapsulated strain composition, the pH strongly decreased over the course of the entire colonic incubation (i.e. from 0 hours to 48 hours). As with the blank/control suspensions, this decrease was especially pronounced between 1 hour and 24 hours of incubation. However, a slight increase was observed during the final 24 hours under fed conditions (FIG. 8A, fourth column, ΔpH=+0.03 from 24 h to 48 h), indicating that the carbohydrate metabolism shifted toward proteolysis. For both fasted and fed conditions, the overall pH decrease in non-sterile incubations was significantly greater than the corresponding blank control (FIG. 8A: ΔpH=−0.60 vs. −0.13 under fasted conditions, −0.67 vs. −0.23 under fed conditions), indicating that the supplemented prebiotic was efficiently fermented by the newly established gut microbial community. As with the blank controls, the pH decrease was more pronounced under fed conditions in non-sterile incubations; without wishing to be bound by any particular theory, the present inventors hypothesize that this may be explained at least partly by higher nutrient availability and at least partly by higher survival rates of the probiotic strains during passage through the upper GIT.

For sterile incubations of the predigested microencapsulated strain composition, the pH decrease was again more pronounced under fed conditions (FIG. 8B, right column, ΔpH=−0.74) than under fasted conditions (FIG. 8B, left column, ΔpH=−0.16), which may again be explained by higher nutrient availability and/or higher probiotic survival rate. Additionally, the pH decrease under both conditions confirms that the probiotic strains were able to ferment the available nutrients, including those of the prebiotic. Interestingly, supplementation of the fed predigested upper GIT suspension resulted in a similar pH decrease in both the sterile incubation (FIG. 8B, right column, ΔpH=−0.74) and the non-sterile incubation (FIG. 8A, fourth column, ΔpH=−0.67), whereas under fasted conditions, the pH decrease was much more pronounced in the non-sterile incubation (FIG. 8A, second column, ΔpH=−0.60) than in the sterile incubation (FIG. 8B, left column, ΔpH=−0.16). As the survival rate of the probiotic strains was much higher under fed conditions, the present inventors conclude, without wishing to be bound by any particular theory, that the higher number of viable probiotic cells in the fed suspension was most likely to colonize the gut microbial community of the donor to a greater extent and therefore contributed more significantly to the metabolic activity of the newly established gut microbial community.

### Lactate Concentration

The human intestine harbors both lactate-producing and lactate-consuming bacteria. Lactic acid bacteria, in particular, produce lactate during primary substrate degradation and decrease the pH of the intestinal environment. Especially at low pH values, lactate can exert a strong antimicrobial effect against pathogens. Another beneficial effect of lactate is that it can be efficiently converted to butyrate and/or propionate; thus, as different microbial species produce or consume lactate, an increase in lactate concentration can result from either or both of increased production and decreased consumption.

Referring now to FIGS. 9A and 9B, for the blank controls, lactate concentration initially increased during the first hour of colonic incubation, indicating that lactate was being produced by the gut microbial community of the donor more quickly than it was being consumed. This increase was, however, very limited under both fasted (FIG. 9A, first column, lactate concentration Δ=+0.14 mM) and fed (FIG. 9A, third column, lactate concentration Δ=+0.20 mM) conditions. As incubation proceeded, lactate levels subsequently decreased (Δ=−0.37 mM under fasted conditions, −0.45 mM under fed conditions), indicating either that lactate production diminished or that lactate was more efficiently converted to other metabolites.

For non-sterile incubations of the predigested microencapsulated strain composition, similar effects were observed, i.e. an initial increase during the first hour of incubation (FIG. 9A, second and fourth columns: Δ=0.20 mM under fasted conditions, 0.72 under fed conditions) was followed by a decrease between 1 hour and 24 hours (Δ=−0.56 mM under fasted conditions, −2.45 mM under fed conditions). Under both fasted and fed conditions, the overall decrease from 0 hours to 48 hours was significantly greater for the microencapsulated strain composition than for the blank control (−0.36 vs. −0.23 under fasted conditions, −1.73 vs. −0.25 under fed conditions). Both the initial increase and the subsequent decrease were more pronounced under fed conditions. As the concentration of prebiotic nutrients were similar under both sets of conditions, the present inventors conclude, without wishing to be bound by any particular theory, that the probiotic strains may both provide an initial boost to lactate production and improve conversion of lactate to other metabolites (e.g. propionate, butyrate) during later stages of incubation.

For sterile incubations of the predigested microencapsulated strain composition, relatively high levels of lactate were produced. This was especially true under fed conditions (FIG. 9B, right column, total lactate concentration Δ=+33.5 mM), which may be linked with higher nutrient availability (originating from the difference in gastric juice composition) and a higher survival rate of the probiotic strains. The strong increase in lactate concentrations indicates that the probiotic strains were able to ferment the available nutrients (originating from both the background medium and the prebiotic composition), resulting in accumulation of lactate. Particularly, unlike in the non-sterile incubations (FIG. 9A, second and fourth columns), no net consumption of lactate was observed during the final 24 hours of incubation under sterile conditions (FIG. 9B), perhaps due to the absence of cross-feeding interactions with propionate- and/or butyrate-producing bacterial strains in the background microbial community.

### Total SCFA Production

SCFA production results from carbohydrate metabolism in the colon and is related to various health effects. The SCFAs produced in the greatest amounts in the colon are acetate, propionate, and butyrate, each of which has a beneficial effect on the health of the host-acetate can be used as an energy source for the host and a potential substrate for lipid synthesis, propionate improves metabolic homeostasis by reducing cholesterol and fatty acid synthesis in the liver, and butyrate is a major energy source for colonocytes and induces differentiation in these cells (an effect that is related to cancer prevention). Branched SCFAs (bSCFAs), by contrast, are a result of proteolytic microbial activity, which is also associated with the formation of toxic compounds such as p-cresol. Thus, high production of acetate, propionate, and butyrate in the colon is associated with beneficial health effects, while high bSCFA production in the colon is associated with detrimental health effects.

Referring now to FIGS. 10A and 10B, total SCFA production reflects the overall fermentation by the gut microbiota. The fasted and fed blank controls (FIG. 10A, first and third columns) resulted in total SCFA production of 32.9 and 44.6 mM, respectively. Thus, the gut microbiota of the donor were able to ferment the nutrients available in the colonic medium. The higher value obtained under fed conditions may again be linked to higher nutrient availability originating from the difference in gastric juice composition. Under both fasted and fed conditions, the strongest SCFA production occurred during the first 24 hours of incubation, which corresponds with the pH decrease observed (FIG. 8A) during this time interval.

For non-sterile incubations of the predigested microencapsulated strain composition, significantly higher total SCFA concentrations (FIG. 10A, second and fourth columns: total SCFA=58.8 mM fasted, 68.0 mM fed) were obtained as compared with the blank controls, with the fed conditions resulting in slightly higher total SCFA production. As the concentration of prebiotic was similar under both conditions, the enhanced production under fed conditions is likely the result of the higher survival rate of microbial strains, which can increase SCFA production either directly (by fermenting available nutrients themselves) or indirectly (by stimulating fermentation of available nutrients by the autochthonous gut microbiota). As with the blank controls, the strongest increase in total SCFA production occurred in the first 24 hours.

For sterile incubations of the predigested microencapsulated strain composition, almost no SCFAs were produced under fasted conditions (FIG. 10B, left column), while under fed conditions the total SCFA production was only 8.5 mM under fed conditions (FIG. 10B, right column). These results confirm that the probiotic strains contribute to SCFA production directly, i.e. converting available nutrients to SCFAs by themselves.

### Acetate Production

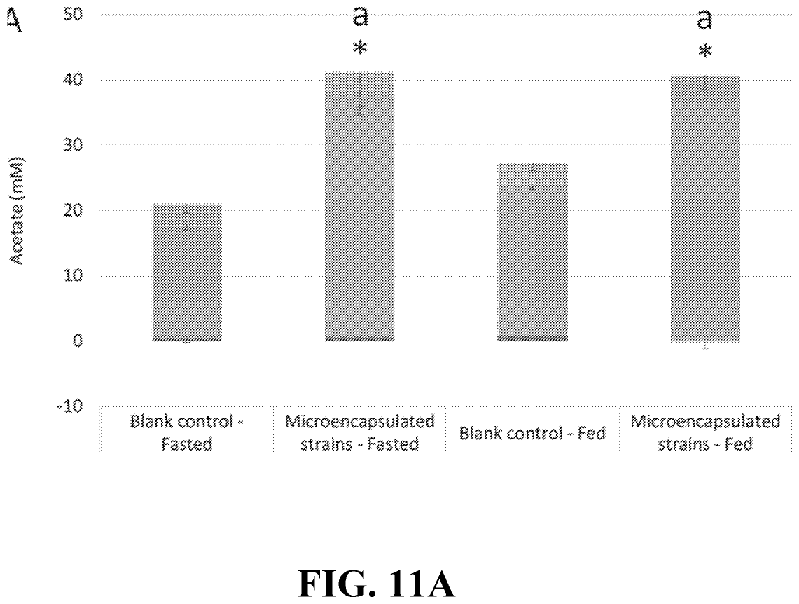

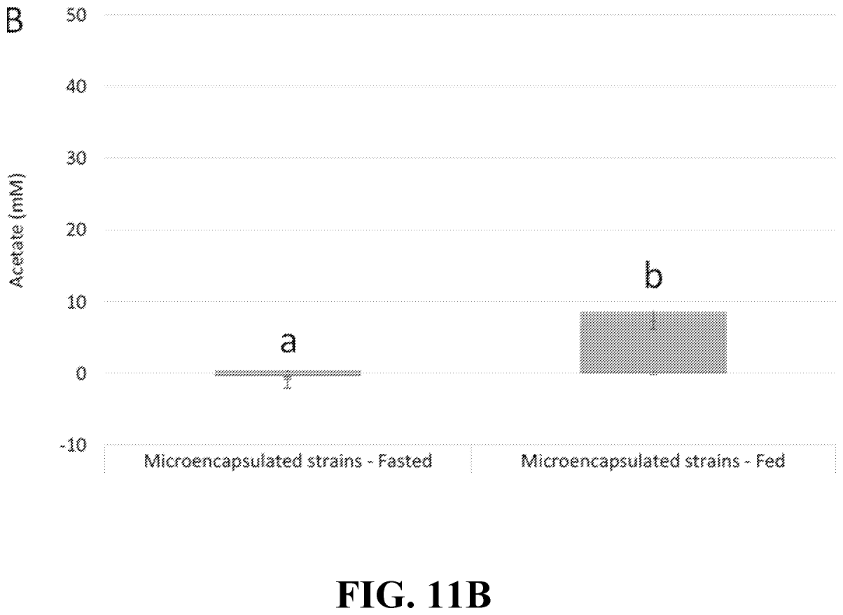

Acetate is one of the key metabolites formed during primary substrate fermentation and can be produced by a wide range of gut microbes, including Bacteroides spp. and Bifidobacterium spp. Referring now to FIGS. 11A and 11B, fasted and fed blank controls (FIG. 11A, first and third columns) resulted in overall acetate production of 21.0 and 27.3 mM, respectively, indicating that total SCFA production largely consisted of acetate. As with total SCFA production (FIG. 10A, first and third columns), higher acetate levels were obtained under fed conditions, and acetate levels increased most markedly during the first 24 hours of incubation.

For non-sterile incubations of the predigested microencapsulated strain composition, significantly higher acetate levels were obtained as compared to the controls under both fasted (FIG. 11A, second column, 41.2 mM) and fed (FIG. 11A, fourth column, 40.6 mM) conditions. No significant difference was observed between the fasted and fed conditions, which the present inventors believe, without wishing to be bound by any particular theory, may indicate that the autochthonous gut microbiota was primarily responsible for acetate production.

For sterile incubations of the predigested microencapsulated strain composition, very little acetate was produced under fasted conditions (FIG. 11B, left column), while under fed conditions the total acetate production was only 8.6 mM (FIG. 10B, right column). Based on these results, the present inventors conclude that the SCFA activity of the probiotic strains results mainly in the production of acetate, which is typical of Lactobacillus and Bifidobacterium strains. The more pronounced increase in acetate concentrations under fed conditions is likely due to higher nutrient availability and higher survival of the probiotic strains exiting the small intestine. Additionally, the much lower acetate levels produced in sterile incubations as compared to non-sterile incubations confirm that administration of the microencapsulated strain composition stimulated the production of acetate by the donor's autochthonous gut microbiota.

### Propionate Production

Like acetate, propionate can be produced by a wide range of gut microbes, with the most abundant propionate producers in the gut microbiome being Bacteroides spp., Akkermansia muciniphila, and Veillonellaceae spp.

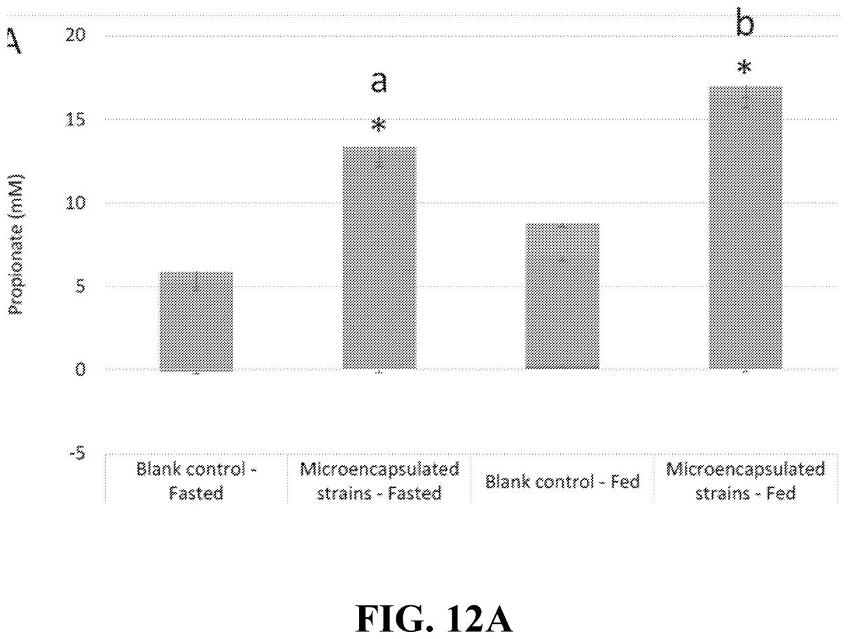

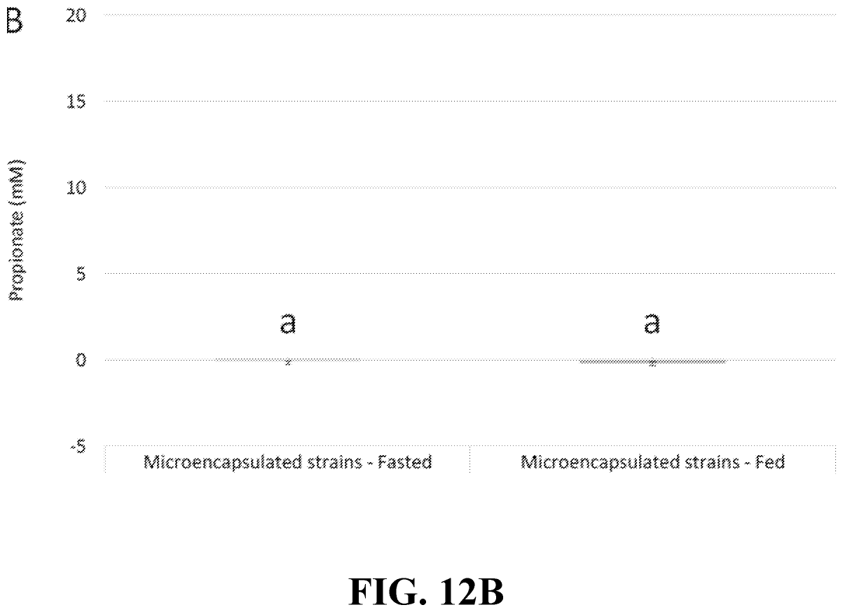

Referring now to FIGS. 12A and 12B, the overall propionate production levels in the blank controls under fasted and fed conditions (FIG. 12A, first and third columns) were 5.8 and 8.8 mM, respectively. Like acetate, propionate was mainly produced during the first 24 hours of incubation and was produced in greater quantity under fed conditions than under fasted conditions. Propionate production thus clearly contributed to total SCFA production in the controls, although to a significantly lesser extent than acetate.

For non-sterile incubations of the predigested microencapsulated strain composition, significantly higher propionate levels were obtained as compared to the blank control (FIG. 12A, second and fourth columns: 13.34 mM under fasted conditions, 16.99 mM under fed conditions). The significantly greater propionate production under fed conditions is again linked with the higher nutrient availability and/or higher survival rate of probiotic strains.

For sterile incubations of the predigested microencapsulated strain composition, the probiotic strains were not able to produce significant amounts of propionate under either fasted (FIG. 12B, left column, 0.00 mM) or fed (FIG. 12B, right column, −0.16 mM) conditions. Based on these results, the present inventors conclude, without wishing to be bound by any particular theory, that at least one of the probiotic microbial strains and the prebiotic composition indirectly increase propionate production by stimulating the autochthonous gut microbial community. This may be the result of a cross-feeding mechanism, whereby the end products of the metabolic activity of certain bacterial species can be used as substrates by other bacterial species. In this case, certain gut microbiota may be able to convert lactate to propionate.

### Butyrate Production

Butyrate is produced by members of Clostridium clusters IV and XIVa. By cross-feeding, these microbes can convert acetate and/or lactate, among other substrates, into butyrate.

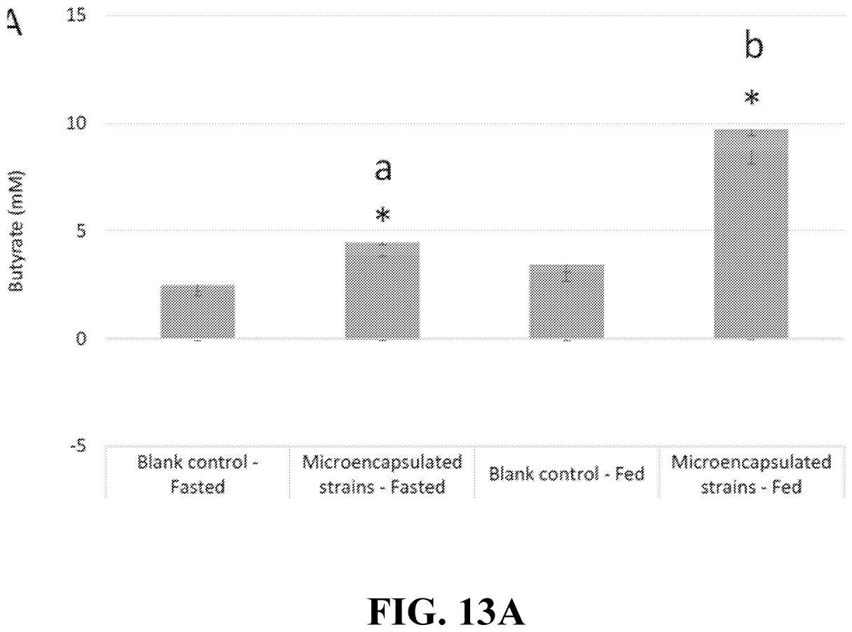

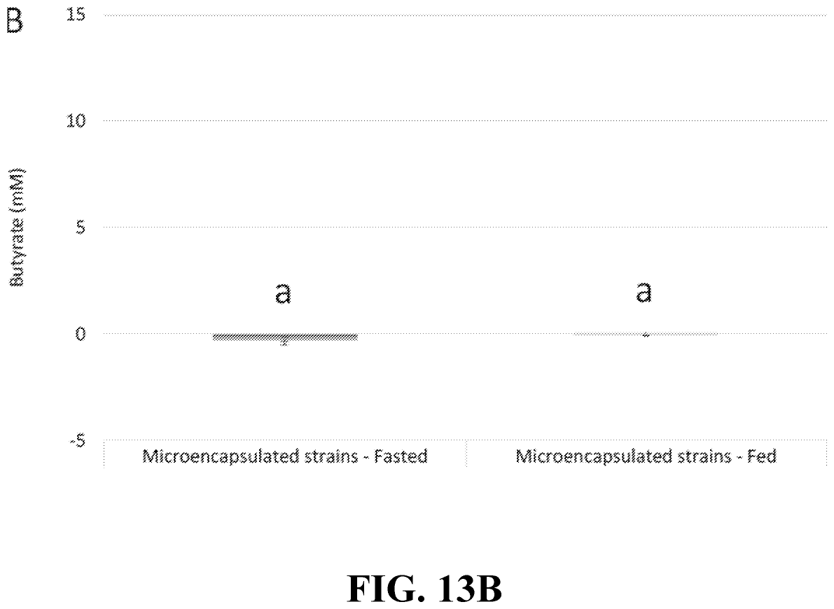

Referring now to FIGS. 13A and 13B, the blank controls (FIG. 13A, first and third columns) resulted in butyrate productions of 2.5 and 3.5 mM, respectively. As with acetate and propionate, butyrate was mainly produced during the first 24 hours of incubation, and slightly higher production was obtained under fed conditions than under fasted conditions.

For non-sterile incubations of the predigested microencapsulated strain composition, significantly higher butyrate levels were obtained as compared to the corresponding blank controls (FIG. 13A, second and fourth columns: 4.41 mM under fasted conditions, 9.70 mM under fed conditions). The highest values were again obtained under fed conditions, which is likely linked to higher nutrient availability and/or higher survival rates of the probiotic strains.

For sterile incubations of the predigested microencapsulated strain composition, the probiotic strains were not able to produce significant amounts of propionate under either fasted (FIG. 13B, left column, −0.30 mM) or fed (FIG. 11B, right column, 0.01 mM) conditions. Based on these results, the present inventors conclude, without wishing to be bound by any particular theory, that at least one of the probiotic microbial strains and the prebiotic composition indirectly increase butyrate production by stimulating the autochthonous gut microbial community. This may be the result of a cross-feeding mechanism; in this case, as with propionate, certain gut microbiota may be able to convert lactate to butyrate.

### Branched SCFAs

Branched SCFAs (bSCFAs) are produced as a result of proteolytic fermentation and are associated with the production of toxic compounds such as p-cresol. Therefore, high bSCFA levels can be indicative of a detrimental proteolytic activity in the colon, and reduced levels are considered beneficial to health.

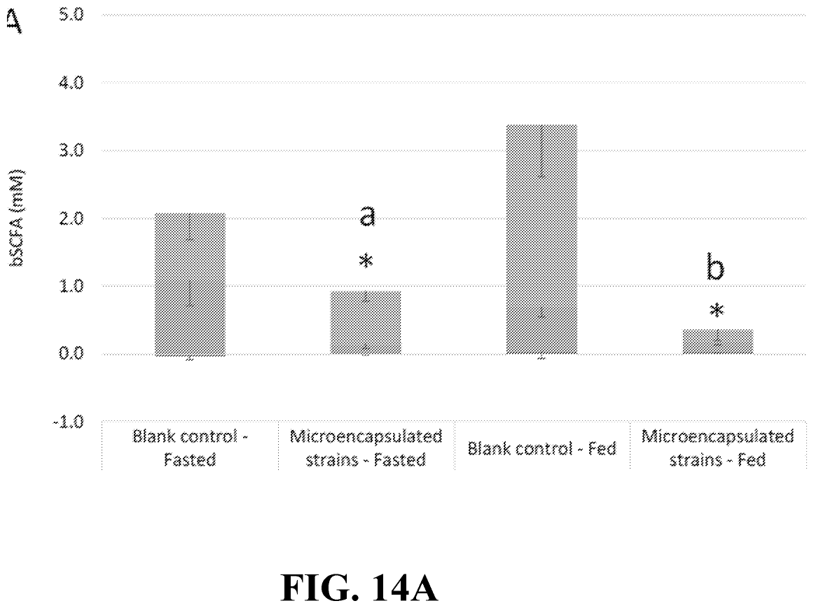

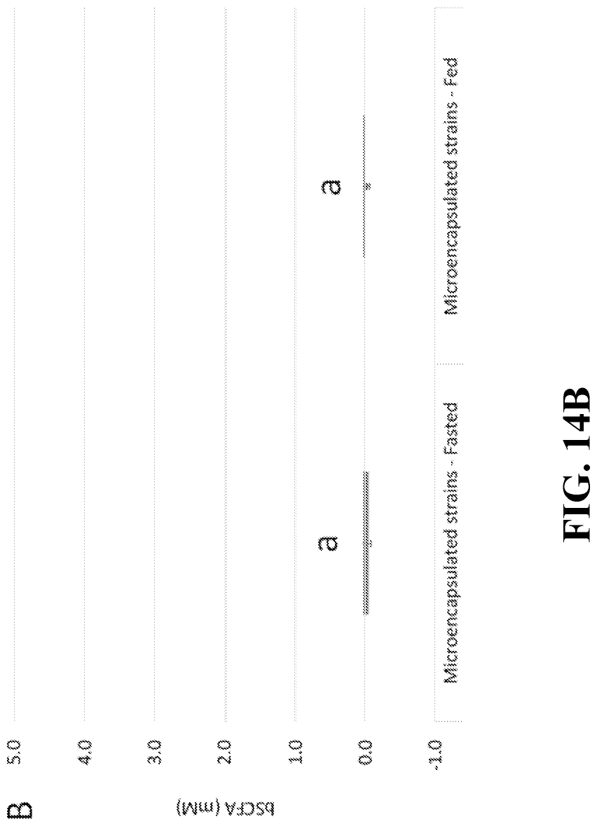

Referring now to FIGS. 14A and 14B, the blank controls, under fasted and fed conditions, resulted in overall bSCFA production of 2.03 and 3.38 mM respectively (FIG. 14A, first and third columns). The bSCFA production was slightly higher under fed conditions, which may again be linked with higher nutrient availability. As compared to acetate, propionate, and butyrate, a distinct increase in bSCFA production is observed during the final 24 hours of incubation, indicating that the gut microbiota initially use carbohydrates as substrates before shifting metabolic activity toward proteolysis later in incubation.

For non-sterile incubations of the predigested microencapsulated strain composition, significantly lower bSCFA concentrations were obtained as compared to the corresponding blank control (FIG. 14A, second and fourth columns: 0.92 mM under fasted conditions, 0.36 mM under fed conditions). Additionally, the lowest values were obtained under fed conditions. Administration of the synbiotic composition thus resulted in increased saccharolytic fermentation, probably due to the presence of the prebiotic.

For sterile incubations of the predigested microencapsulated strain composition, the probiotic strains were not able to produce significant amounts of bSCFA under either fasted (FIG. 14B, left column, 0.00 mM) or fed (FIG. 14B, right column, 0.03 mM) conditions.

### Example 5

### Comparative Survival of Probiotic Strains Along Upper GIT

Survival of microbial strains in commercially available pediatric probiotic products was investigated by subjecting the probiotic products to treatment in the adapted SHIME system 500 illustrated in FIG. 4, as described above in Example 3. The probiotic products were added to the reactor vessel 505 at the start of the upper GIT experiment under fed conditions in amounts corresponding to the recommended daily dosage of each product. Four commercially available pediatric probiotic products were tested: a Culturelle® Kids Probiotic composition (1 sachet/reactor), a Garden of Life® Raw Probiotics Kids composition (1.6 g/reactor), an Optibac Probiotics® Babies & Children composition (1 sachet/reactor), and a Life-Space® Probiotic Powder for Children composition (1 g/reactor).

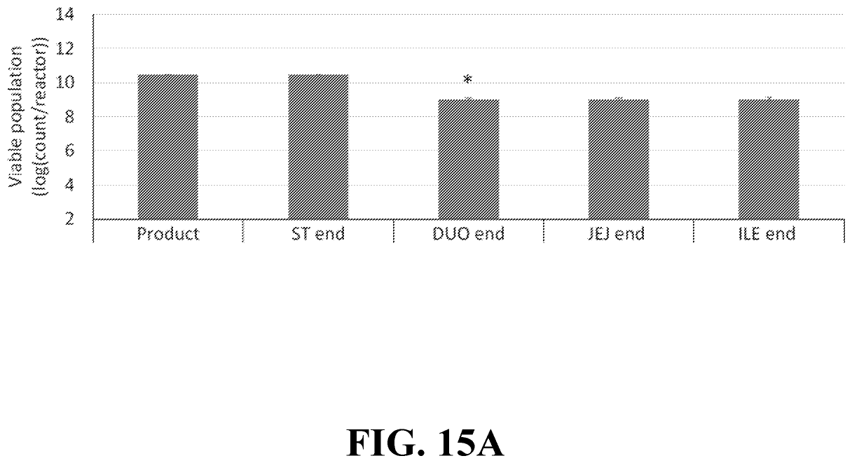

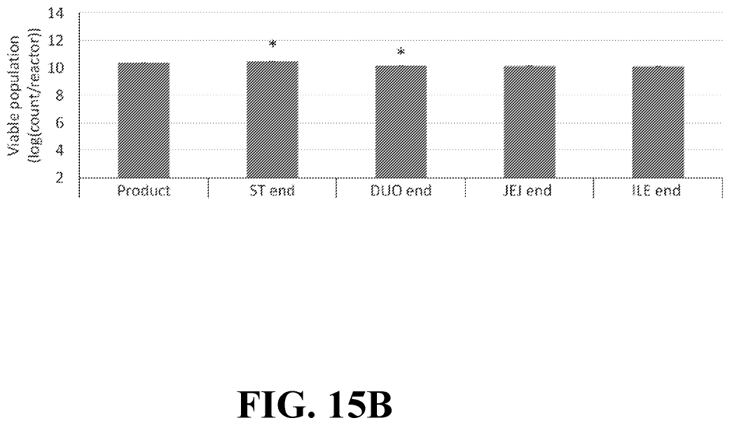

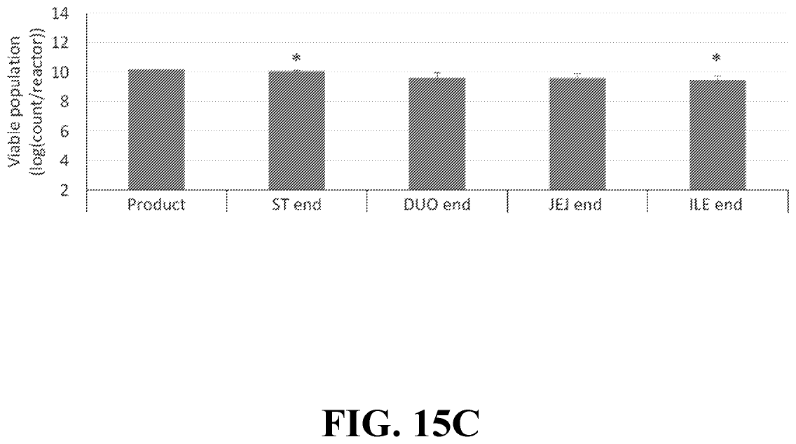

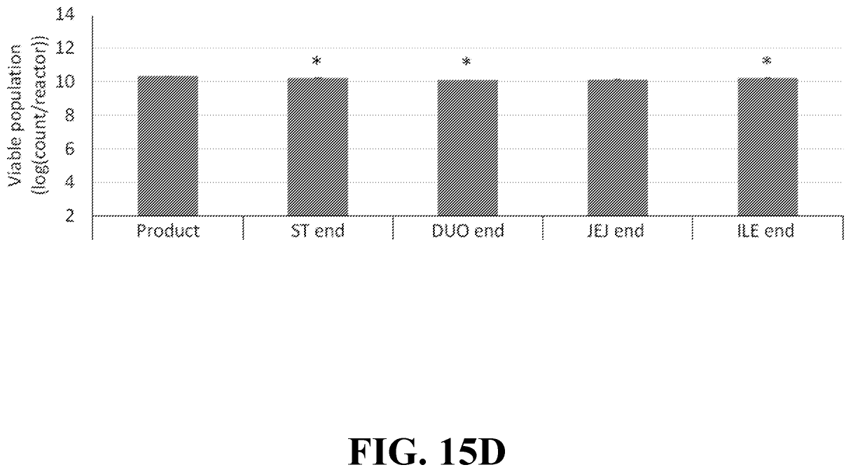

Referring now to FIG. 15A, administration of the Culturelle® Kids Probiotic composition, whose microbial strains consist primarily of Lactobacillus rhamnosus GG cells, revealed that the viability of the probiotic strain was largely unaffected during passage through the fed stomach (10^{10.47} viable cells per reactor at both the beginning and end of the stomach conditions). The Lactobacillus rhamnosus GG strain present in this probiotic composition (a) thus proved to be resistant to the pH profile (i.e. the sigmoidal decrease in pH from 4.6 to 3.0 over 2 hours) in the stomach. Upon entering the duodenal phase, however, the viability of the probiotic strain significantly decreased (from 10^{10.47}=2.97·10¹⁰ to 10^{9.02}=1.06·10⁹ viable cells per reactor); as described above in Example 3, bile salts added at the beginning of the duodenal phase can be highly toxic to bacterial cells, especially at relatively low pH. As the pH increased throughout the jejunal and ileal phases, no further reduction in viability was observed (10^{9.02} and 10^{9.03} viable cells at the end of the jejunal and ileal phases, respectively). Thus, from the beginning to the end of the upper GIT, the number of viable cells was reduced by 96.27% (survival of 3.73%), from 10^{10.47}=2.95·10¹⁰ to 10^{9.03}=1.10·10⁹ viable cells per reactor.

Referring now to FIG. 15B, administration of the Garden of Life® Raw Probiotics Kids composition, whose microbial strains consist primarily of Lactobacillus spp. and Bifidobacterium spp., revealed that the microbial strains present in the probiotic product were not negatively affected by the environmental conditions encountered during transit through the stomach under fed conditions. In fact, at the end of transit through the stomach, the viable cell count was significantly higher than at the beginning (10^{10.48}=3.02·10¹⁰ vs. 10^{10.37}=2.36·10¹⁰ viable cells per reactor), although the in vivo implications of this finding may be questionable; growth of probiotic strains during transit through the stomach is quite unlikely, and the measured effect may therefore be an artifact of the lower solubility of the test composition in aqueous buffer compared to the gastric juice simulation medium. The viability of the probiotic strains was then significantly reduced (to 10^{10.16}=1.45·10¹⁰ viable cells per reactor) in the duodenal phase, which again may be linked to the toxicity of bile salts at low pH. No significant reductions in viability were observed in the jejunal and ileal phases. At the end of the ileal phase, a viable population density of 10^{10.13}=1.35·10¹⁰ viable cells per reactor was observed, representing a decrease of 42.82% (survival of 57.18%) as compared to the initial population and 55.33% (survival of 44.67%) when compared to the viable population at the end of the stomach phase.

Referring now to FIG. 15C, administration of the Optibac Probiotics® Babies & Children composition, whose microbial strains consist primarily of Lactobacillus acidophilus Rosell-52, Bifidobacterium infantis Rosell-33, and Bifidobacterium bifidum Rosell-71, revealed that the viability of the strains was negatively affected during passage through the stomach, with a significant reduction from 10^{10.20}=1.59·10¹⁰ to 10^{10.08}=1.20·10¹⁰ viable cells per reactor. This indicates that these strains are less robust when faced with the harsh pH conditions in the stomach. The viable population density further decreased throughout the small intestinal phases, although it was only by the end of the ileal phase that this reduction (to 10^{9.48}=3.49·10⁹ viable cells per reactor) was statistically significant. Thus, these strains were likely also negatively affected by the high bile salt concentrations and low pH values in the duodenal phase, albeit to a lesser extent than for the Culturelle® Kids Probiotic composition (FIG. 15A). Overall, passage through the upper GIT resulted in a viability reduction of 78.04% (survival of 21.96%) for this composition.

Referring now to FIG. 15D, administration of the Life-Space® Probiotic Powder for Children composition, whose microbial strains consist primarily of Lactobacillus spp. and Bifidobacterium spp., revealed that the viability of these strains was negatively affected during passage through the stomach, with a significant reduction in viable cell count from 10^{10.32}=2.07·10¹⁰ to 10^{10.21}=1.63·10¹⁰ viable cells per reactor. As with the Optibac Probiotics® Babies & Children composition (FIG. 15C), this reduction was most likely the result of poor robustness of the probiotic strains against the harsh pH conditions in the stomach. The viable population density decreased further in the duodenal phase (to 10^{10.08}=1.21·10¹⁰ viable cells per reactor), indicating that these strains were also negatively affected by the addition of bile salts. However, no further reduction in viability was observed in the jejunal phase, and in fact an increase in viability was observed in the ileal phase (from 10^{10.10}=1.26·10¹⁰ viable cells per reactor at the end of the jejunal phase to 10^{10.21}=1.61·10¹⁰ viable cells pre reactor at the end of the ileal phase). Overall, passage through the upper GIT resulted in a viability reduction of 22.31% (survival of 77.69%) for this composition.

A comparison of viable cell survival rates after transit through the upper GIT for the four commercially available compositions tested in this Example and the two compositions (the naked strain composition and the microencapsulated strain composition) of the present disclosure tested in Example 3 is provided in the below Table 2. In Table 2, the survival rate is simply the number of colony forming units (CFU) obtained at the end of the ileal phase divided by the number of CFUs present in the probiotic composition at the outset of the experiment, “MXP271+” refers to the compositions of the present disclosure tested in Example 3 (i.e. the PDS-08 strains, comprising the seven MXP271 strains plus Lactobacillus acidophilus SD-NCFM-US and Bifidobacterium animalis subsp. lactis SD-BI07-US), and “ME” means “microencapsulated.”

TABLE 2

| | Upper GIT | | |
|---|---|---|---|
| | survival | | |
| Probiotic | rate | Viable cells after ileal phase | |
| composition | (%) | Log₁₀(CFU/reactor) | CFU/reactor |
| Culturelle | 3.73 | 9.03 | 1.10 · 10⁹ |
| Garden of Life | 57.18 | 10.13 | 1.35 · 10¹⁰ |
| Optibac | 21.96 | 9.48 | 3.49 · 10⁹ |
| Life-Space | 77.69 | 10.21 | 1.61 · 10¹⁰ |
| MXP271+ (naked) | 19.33 | 10.19 | 1.57 · 10¹⁰ |
| MXP271+ (ME) | 35.73 | 10.43 | 2.72 · 10¹⁰ |

As illustrated in Table 2, survival rates of the Culturelle® Kids Probiotic composition are generally too low to provide a sufficiently high number of viable cells to the site of their probiotic activity (i.e. the distal ileum and colon). While a similar proportion of the cells of the Optibac Probiotics® Babies & Children composition survive the upper GIT as in the naked strain composition of the present disclosure, the lower number of cells provided in the recommended dosage form results in a much smaller number of viable cells being delivered to the distal ileum and colon. The higher initial viable cell density in the naked strain composition also resulted in a number of viable cells at the end of the ileal phase higher than that of the Garden of Life® Raw Probiotics Kids composition and comparable to that of the Life-Space® Probiotic Powder for Children composition. Finally, the microencapsulated strain composition of the present disclosure delivered a noticeably larger number of viable cells to the end of the ileal phase than any of the currently commercially available pediatric probiotic compositions that were tested.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1. A method for treating a disease or achieving a physiological objective in a human subject, comprising administering to the subject a therapeutically effective amount of a synbiotic composition, the synbiotic composition comprising:
a prebiotic component, comprising at least one compound that can be converted, by a microbial strain present in the healthy human gut microbiota, into a bioactive metabolite; and
a probiotic component, comprising a consortium of microbial strains, the consortium comprising at least two microbial strains selected from the group consisting of:
(i) Lacticaseibacillus rhamnosus SD-GG-BE;
(ii) Ligilactobacillus salivarius SD-LS1-IT;
(iii) Bifidobacterium breve SD-BR632-IT;
(iv) Bifidobacterium breve SD-BR03-IT;
(v) Bifidobacterium longum SD-CECT7347-SP;
(vi) Lacticaseibacillus casei SD-CECT9104-SP; and
(vii) Bifidobacterium lactis SD-CECT8145-SP.

2. The method of claim 1, wherein the disease is selected from the group consisting of adrenal leukodystrophy, AGE-induced genome damage, Alexander Disease, alopecia areata, Alper's Disease, Alzheimer's disease, amyotrophic lateral sclerosis, angina pectoris, arthritis, asthma, Balo's concentric sclerosis, Behcet's disease, bullous pemphigoid, Canavan disease, cardiac insufficiency including left ventricular insufficiency, central nervous system vasculitis, Charcot-Marie-Tooth Disease, childhood ataxia with central nervous system hypomyelination, chronic idiopathic peripheral neuropathy, chronic obstructive pulmonary disease, Crohn's disease, cutaneous lupus, dermatitis (contact, acute and chronic), diabetic retinopathy, graft versus host disease, granulomas, hepatitis C viral infection, herpes simplex viral infection, herpes zoster viral infection, human immunodeficiency viral infection, Huntington's disease, irritable bowel disorder, ischemia, Krabbe Disease, lichen planus, macular degeneration, mitochondrial encephalomyopathy, monomelic amyotrophy, multiple sclerosis, myocardial infarction, neurodegeneration with brain iron accumulation, neuromyelitis optica, neurosarcoidosis, NF-κB mediated diseases, optic neuritis, paraneoplastic syndromes, Parkinson's disease, Pelizaeus-Merzbacher disease, pemphigus, primary lateral sclerosis, progressive supranuclear palsy, psoriasis, pyoderma gangrenosum, reperfusion injury, retinitis pigmentosa, sarcoidosis, Schilder's Disease, subacute necrotizing myelopathy, susac syndrome, transplantation rejection, transverse myelitis, a tumor, ulcerative colitis, and Zellweger's syndrome.

3. The method of claim 1, wherein the disease is a gastroenterological or infectious disease.

4. The method of claim 3, wherein the disease is selected from the group consisting of irritable bowel syndrome, COVID-19, and constipation.

5. The method of claim 3, wherein the disease is antibiotic-induced dysbiosis of the subject's gut microbiota.

6. The method of claim 1, wherein the disease is selected from the group consisting of metabolic syndrome, type 2 diabetes, and pre-diabetes.

7. The method of claim 1, wherein the physiological objective is selected from the group consisting of improving cardiovascular health, maintaining or reducing body weight, reducing glycated hemoglobin levels, normalizing glucose or insulin response, treating acne or otherwise improving skin health, and improving cognitive function.

8. The method of claim 1, further comprising:
co-administering, or administering in combination with the synbiotic composition, one or more other pharmaceutical formulations to a human subject,
wherein the co-administration or administration in combination improves a therapeutic effect of the one or more other pharmaceutical formulations relative to independent administration of the synbiotic composition and the one or more other pharmaceutical formulations.

9. The method of claim 1, wherein the physiological objective is selected from the group consisting of increasing Bristol Stool Form Scale (BSFS), decreasing duration of a bowel movement, relieving abdominal pain, relieving bloating, relieving heartburn, relieving acid reflux, relieving indigestion, maintaining or increasing diversity of the gastrointestinal microbiota, and improving health-related quality of life as measured by KINDL questionnaire score.

10. The method of claim 1, wherein the synbiotic composition is administered as an ingestible formulation.

11. The method of claim 10, wherein the ingestible formulation is in the form of a free-flowing powder provided in a single-serving sachet.

12. The method of claim 11, wherein the sachet comprises the prebiotic component in an amount of from about 1 mg to about 12 g, or from about 250 mg to about 11.75 g, or from about 500 mg to about 11.5 g, or from about 750 mg to about 11.25 g, or from about 1 g to about 11 g, or from about 1.25 g to about 10.75 g, or from about 1.5 g to about 10.5 g, or from about 1.75 g to about 10.25 g, or from about 2 g to about 10 g, or from about 2.25 g to about 9.75 g, or from about 2.5 g to about 9.5 g, or from about 2.75 g to about 9.25 g, or from about 3 g to about 9 g, or from about 3.25 g to about 8.75 g, or from about 3.5 g to about 8.5 g, or from about 3.75 g to about 8.25 g, or from about 4 g to about 8 g, or from about 4.25 g to about 7.75 g, or from about 4.5 g to about 7.5 g, or from about 4.75 g to about 7.25 g, or from about 5 g to about 7 g, or from about 5.25 g to about 6.75 g, or from about 5.5 g to about 6.5 g, or from about 5.75 g to about 6.25 g, or about 6 g.

13. The method of claim 11, wherein the sachet comprises the consortium of microbial strains in an amount of from about 62.5 million AFU to about 312.5 billion AFU, from about 625 million AFU to about 250 billion AFU, from about 1.25 billion AFU to about 125 billion AFU, from about 6.25 billion AFU to about 62.5 billion AFU, from about 12.5 billion AFU to about 60 billion AFU, from about 25 billion AFU to about 55 billion AFU, from about 40 billion AFU to about 50 billion AFU, or about 45 billion AFU.

14. The method of claim 11, wherein a material of the sachet has a moisture vapor transmission rate, at 23° C. and 50% relative humidity, of less than about 0.01 grams per square meter per day.

15. The method of claim 11, wherein a dose of the synbiotic composition is administered at least once per day, wherein a dose comprises the contents of one sachet.

16. The method of claim 11, wherein the sachet further comprises at least one pharmaceutically acceptable vehicle.

17. The method of claim 1, wherein the synbiotic composition is administered at least once per day for at least about 7 days.

18. The method of claim 1, wherein the at least one compound that can be converted, by a microbial strain present in the healthy human gut microbiota, into a bioactive metabolite comprises at least one fructan.

19. The method of claim 18, wherein the at least one fructan comprises at least one polysaccharide fructan.

20. The method of claim 19, wherein the at least one polysaccharide fructan comprises an inulin.

21. The method of claim 20, wherein the inulin is derived or extracted from at least one plant selected from the group consisting of agave, asparagus, banana, barley, burdock, camas, chicory, coneflower, costus, dandelion, elecampane, garlic, globe artichoke, Jerusalem artichoke, jicama, leek, leopard's bane, mugwort, onion, plantain, wheat, yacón, and yam.

22. The method of claim 18, wherein the at least one fructan comprises a fructooligosaccharide.

23. The method of claim 22, wherein the fructooligosaccharide is derived or extracted from at least one plant selected from the group consisting of agave, asparagus, banana, barley, burdock, camas, chicory, coneflower, costus, dandelion, elecampane, garlic, globe artichoke, Jerusalem artichoke, jicama, leek, leopard's bane, mugwort, onion, plantain, wheat, yacón, and yam.

24. The method of claim 1, wherein the prebiotic component consists essentially of about 50 wt % inulin and about 50 wt % fructooligosaccharides.

25. The method of claim 1, wherein the consortium comprises at least three, at least four, at least five, at least six, or all of (i) through (vii).

26. The method of claim 1, wherein the consortium further comprises at least one microbial strain selected from the group consisting of:
(viii) Lactobacillus acidophilus SD-NCFM-US; and
(ix) Bifidobacterium animalis subsp. lactis SD-BI07-US.

27. The method of claim 26, wherein the consortium comprises both of (viii) and (ix).

28. The method of claim 27, wherein the consortium comprises all of (i) through (vii) and both of (viii) and (ix).

29. The method of claim 28, wherein the consortium consists essentially of (i) through (vii), (viii), and (ix).
